(19) United States
(12) Patent Application Publication
(10) Pub. No.: US 2006/0014191 A1
(43) Pub. Date: Jan. 19, 2006

(54) Analog probe complexes

(76) Inventors: Kai Qin Lao, Pleasanton, CA (US); Timothy G. Geiser, San Mateo, CA (US); Neil A. Straus, Emeryville, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/172,280

(22) Filed: Jun. 29, 2005

Related U.S. Application Data
(60) Provisional application No. 60/584,799, filed on Jun. 30, 2004.

(51) Int. Cl. C12Q 1/68; C07K 14/47

(52) U.S. Cl. 435/6; 530/350

(57) ABSTRACT

The present invention relates to the detection of target sequences. The present description discloses compositions and methods involving analog nucleic acids, such as PNA and L-DNA, for the detection of nucleic acids. Additionally, hybrid detectable markers are provided.

### CROSS REFERENCE

This application claims priority under 35 U.S.C. §119(e) to U.S. Provisional Patent Application Ser. No. 60/584,799, filed on Jun. 30, 2004, hereby incorporated by reference in its entirety.

### FIELD

The invention relates to the field of probe-based nucleic acid sequence detection, analysis and quantitation. In particular, the invention relates to various novel compositions and methods relating to probes.

### INTRODUCTION

Despite considerable progress in transcription and translational profiling with gene and protein microarrays, methods and compositions that continuously monitor gene expression dynamics in live cells are in high demand. In addition, current microarray technologies cannot detect low copy number gene products, which often play a prominent role in sensing, signaling and gene regulation. One possible method for achieving this goal is through the use of single-molecule detection.

In recent years, progress has been made in the area of single-molecule detection in biological systems. The single-molecule approach has changed the way many biological problems are addressed and interpreted. (Xie et al., Ann. Rev. Phys. Chem., 49: 441-480 (1998); Weiss, S., Science, 283(5408):1676-83 (1999); Sosa et al., Nat Struct Biol, 8(6): 540-4 (2001); Zhuang et al., Proc Natl Acad Sci USA, 97(26):14241-4 (2000); Moerner et al., Science, 283(5408):1670-6 (1999)). New insights derived from this approach are continuing to emerge. Although most of the single-molecule work has been carried out in vitro, single-molecule experiments in living cells are beginning to appear. (Sako et al., Cell Struct Funct, 27(5):357-65 (2002); Sako et al., Nat Cell Biol, 2(3):168-72 (2000); Seisenberger et al., Science, 294(5548):1929-32 (2001); Harms et al., Biophys J, 81(5):2639-46 (2001)).

Typically, techniques for mRNA detection, such as Northern hybridization and fluorescence in situ hybridization (FISH), have been performed in vitro or in fixed cells. Real time imaging in live cells became possible only recently (Sokol et al., Proc Natl Acad Sci USA, 95(20):11538-43 (1998); Perlette et al., Anal Chem, 73(22):5544-50 (2001)) with the emergence of various fluorescent probe techniques. One type of fluorescent probe is the molecular beacon. (Tyagi et al., Nat Biotechnol, 14(3):303-8 (1996)).

FIG. 1 displays one example of a molecular beacon. This particular molecular beacon is a hairpin-shaped single stranded DNA with the ends labelled with a fluorophore and a nonfluorescent quencher, respectively. In the absence of target mRNA, the beacon adopts a closed conformation, in which the emission of the fluorophore is quenched. In the presence of target mRNA, the beacon hybridizes to the complementary sequence, forcing the beacon to open and restore the fluorescence. This feature allows the beacons to report target molecules in real time, eliminating the need to wash away unhybridized probes from the reaction mixture before signal collection. However, these DNA beacons are subject to degradation by various DNA nucleases. Li et al., (Nucleic Acids Res, 28(11):E52 (2000)). Also various DNA binding proteins can cause the beacons to open and give false fluorescent signal. Li et al., (Andew Chem Int Ed Engl, 39(6):1049-1052 (2000)). For these reasons, as well as others, the DNA beacons are not desirable for imaging in live cells, which contain various nucleases and DNA binding proteins.

Some progress has recently been made to circumvent this difficulty with PNA molecular beacons. (Ortiz et al., Mol Cell Probes, 12(4):219-26 (1998); Seitz, O., Angew Chem Int Ed Engl, 39(18):3249-3252 (2000); Kuhn et al., Antisense Nucleic Acid Drug Dev, 11(4):265-70 (2001); Kuhn et al. J Am Chem Soc, 124(6):1097-103 (2002)). PNA is a DNA analogue in which the nucleotides are attached to a pseudo-peptide backbone (Nielsen et al., Bioconjug Chem, 5(1):3-7 (1994); Hyrup et al., Bioorg Med Chem, 4(1):5-23 (1996)(see FIG. 2 of Hyrup et al.)). PNA hybridizes with complementary DNA, mRNA or PNA oligomers through Watson and Crick base pairing.

The peptide backbone of PNA provides resistance to degradation by nuclease, and is accessible to a variety of chemical modifications. (Nielsen et al., Curr Issues Mol Biol, 1(1-2):89-104 (1999); Ray et al., Faseb J, 14(9):1041-60 (2000)). Like DNA beacons, PNA beacons are made by linking a fluorophore and a quencher to the two ends of the random coil of PNA. However, unlike DNA beacons, the traditional PNA beacon does not rely on hairpin formation. This is because the PNA hairpin is too stable to open in order to allow hybridization with mRNA. The existing PNA beacons are generally stemless and in a random coil conformation, which results in a lower signal to background ratio (SN/) of ˜10, compared to ˜25 for the DNA beacons. (Tyagi et al., Nat Biotechnol, 14(3):303-8 (1996), and see U.S. Pat. No. 6,607,889, issued Aug. 19, 2003 to Coull et al for other compositions of PNA beacons).

The earliest description of a hybridization sensitive fluorescent probe was by Morrison et al. (Anal. Biochem. 183:231-244, (1989)). Subsequent papers have described hair-pin “beacon” probes (e.g., Tyagi and Kramer, Nat. Biotech. 14:303-308 (1996)) and PNA/DNA beacons (e.g., Ortiz et al. Molec Cellular Probes 12:219-226 (1998)). Additionally, U.S. Patent Publication from Coull et al., further describes various PNA molecular probes (U.S. Patent Publication 2003/0036059, published Feb. 20, 2003), as does U.S. Pat. No. 6,607,889, (issued to Coull et al, Aug. 19, 2003), both involving PNA segments hybridized to natural D-DNA segments. Other nonhybridized chimeric probes involving enantiomeric versions of DNA, have been discussed as single probe options in Greenfield et al., Pub. No. 20030198980, published Oct. 23, 2003.

### SUMMARY

In one embodiment, an analog probe complex for the detection of nucleic acids is provided. The analog probe complex comprises a first nucleic acid analog segment comprising a Protein Nucleic Acid (PNA) segment that hybridizes to a first target sequence and a second nucleic acid analog segment. The first and second segments are configured to effectively hybridize to one another, until the first segment hybridizes to the target sequence. In another embodiment, the analog probe complex comprises L-DNA, L-RNA, LNA, iso-C nucleic acid, iso-G nucleic acid, or any combination thereof. In another embodiment, the analog probe complex comprises L-DNA. In one embodiment, the first nucleic acid analog segment is longer than the second nucleic acid analog segment. In one embodiment the second nucleic acid analog segment will not substantially bind to the first target sequence. In one embodiment, the first nucleic acid analog segment further comprises a detectable marker and the second nucleic acid analog segment further comprises a marker modifier. In another embodiment, the second nucleic acid analog segment further comprises a first fluorescent moiety. In another embodiment, the first nucleic acid analog segment further comprises a second fluorescent moiety. In another embodiment, the fluorescent moieties are configured to result in a fluorescent interaction when the first and second segments are hybridized to one another. In another embodiment, the fluorescent moiety on the first segment is configured to be fluorescent when the fluorescent moiety on the second moiety on the second segment is not in proximity to said first fluorescent moiety. In another embodiment, the fluorescent moiety on the first segment is a fluorescent emitter. In some embodiments, the fluorescent emitter is selected from Quantum dots, Texas red, terbium chelate, europium cryptate, DABCYL, Fluorescein, IAEDANS, EDANS, BODIPY FL, and any combination thereof. In another embodiment, the fluorescent moiety on the second segment is a fluorescence quencher. In another embodiment, the quencher is selected from TRITC (tetrarhodamine isothiocyanate), Allophycocyanin, EDANS, Tetramethylrhodamine, DABCYL, Fluorescein, BODIPY FL, QSY 7 dye, and any combination thereof. In another embodiment, the emitter and quencher are configured so that a first amount of FRET occurs between the emitter and quencher when the first PNA and second nucleic acid analog segments are hybridized to one another, and the first amount of FRET decreases when the two segments are not hybridized to one another. In another embodiment, the emitter is attached to the 5′ prime end of the first nucleic acid analog segment and the quencher is attached to the 3′ end of the second nucleic acid analog segment; thus, placing the emitter and quencher at the same end of the annealed PNA/DNA analog probe.

In another aspect, a L-DNA/PNA analog probe complex is provided. The L-DNA/PNA analog probe complex comprises a first segment comprising a protein nucleic acid (PNA) segment. A fluorescent marker is attached to the first segment. The PNA segment comprises a first PNA probe sequence that is capable of effectively binding to a target sequence. The L-DNA/PNA analog probe complex further comprises a second segment comprising a left-handed DNA (L-DNA) sequence that is capable of dissociably binding to said PNA probe sequence. In another embodiment, the first segment further comprises a second PCA sequence that is configured to not hybridize to the L-DNA sequence, wherein said second PNA sequence binds to the target sequence. In another embodiment, the first and second segments have the same number of nucleotides. In another embodiment, the first and the second segments are bound together via the hybridization of the first PNA sequence and the L-DNA sequence. In another embodiment, the second sequence and the L-DNA sequence are effectively complementary sequences. In another embodiment, the L-DNA/PNA analog probe complex described above further comprises a first fluorescent moiety on the first segment (which can be the fluorescent marker) and a second fluorescent moiety on the second segment, wherein hybridization of the first and second segments results in a first fluorescent signature from the first and second moieties, and a second fluorescent signature is obtained from the first and second moieties in the absence of hybridization. In another embodiment, the first PNA sequence binds to a target sequence that is a RNA sequence. In another embodiment, the first segment is longer in length than the second segment on an end of the first segment distal to the fluorescent moiety. In another embodiment, the nucleotides in the L-DNA segment have a conformation of 1′S, 3′R, and 4′S.

In another aspect, a method of detecting the presence of a nucleic acid sequence in a sample is provided. The method comprises a) contacting an analog probe complex with a sample, the analog probe complex comprises a first section that comprises a protein nucleic acid (PNA) segment and a fluorescent marker. The first section further comprises a first PNA sequence that is capable of effectively binding to a target sequence, and a second PNA sequence and a second section that comprises a left handed DNA (L-DNA) sequence and a fluorescent quencher, wherein the L-DNA sequence is capable of dissociably binding to the second PNA sequence, b) allowing a dissociation of the second sequence from the L-DNA sequence, and c) measuring a fluorescence of the resulting composition, wherein a change in fluorescence indicates the presence of a nucleic acid sequence. In another embodiment, the method further comprises a first step of measuring the fluorescence of the fluorescent marker when the first segment and the second segment are hybridized at the second PNA sequence and the L-DNA sequence, and using this fluorescence to determine if there has been a change in fluorescence. In another embodiment, the target sequence is a RNA sequence. In another embodiment, the target sequence is a DNA sequence. In another embodiment, the fluorescent marker is a quantum dot. In another embodiment, the fluorescent quencher is a black hole quencher. In another embodiment, the target sequence is a product from in vitro transcription. In another embodiment, the detectable marker is a superparamagnetic nanoparticle and the marker modifier is a beta-field shielder. In another embodiment, the superparamagnetic particle and the beta-field shielder are made from a same material. In another embodiment, the superparamagnetic particle comprises Fe₂O₃. In another embodiment, the superparamagnetic particle comprises Gadolinium.

In another aspect, a method of detecting the presence of a nucleic acid sequence is provided. The method comprises a) contacting an analog probe complex with a sample, the analog probe complex comprises a first section that comprises a protein nucleic acid (PNA) and a detectable marker, wherein the first section comprises a first PNA sequence that is capable of effectively binding to a target sequence, and a second PNA sequence; and a second section that comprises a left handed DNA (L-DNA) sequence and a marker modifier, wherein said L-DNA comprises a third sequence that is capable of dissociably binding to said first section, and wherein said second PNA sequence is capable of dissociably binding to said second section, b) allowing a dissociation of the second sequence from the third sequence, and c) monitoring a change in a signal from the detectable marker; the change in the signal of the detectable marker indicates the presence of said nucleic acid sequence. In one embodiment, the detectable marker is a superparamagnetic nanoparticle and wherein the marker modifier is a beta-field shielder. In another embodiment, the monitoring of the change in the signal of the detectable marker is achieved through a MRI device. In another embodiment, the analog probe complex is administered to a patient. In another embodiment, the detection of the change in the signal is done while the detectable marker is in the patient.

In one aspect, a zip-coded analog probe complex is provided. The zip-coded analog probe complex comprises a first segment comprising a first zip-coded L-DNA sequence and a first PNA sequence, wherein the first zip-coded L-DNA sequence and the first PNA sequence are associated and wherein the PNA sequence will effectively hybridize to a target sequence. In another embodiment, the zip-coded analog probe complex further comprises a detectable marker and a second segment that comprises a second zip-coded L-DNA sequence, the second zip-coded L-DNA sequence will effectively hybridize to said first zip-coded L-DNA sequence, and the second zip-coded L-DNA sequence is associated with the detectable marker. In another embodiment, the detectable marker is a fluorescent marker. In another embodiment, the detectable marker is a Quantum dot.

In another aspect, a set of zip-coded analog probe complexes is provided. The set of zip-coded L-DNA analog probe complexes comprises a first zip-coded L-DNA analog probe complex that comprises a) a first segment that comprises a first PNA sequence, wherein the PNA sequence will effectively hybridize to a first target sequence, and a first zip-coded L-DNA sequence, the first zip-coded L-DNA sequence being associated with the PNA sequence, b) a first detectable marker; and c) a second segment that comprises a second zip-coded L-DNA sequence, wherein the second zip-coded L-DNA sequence will effectively hybridize to the first L-DNA sequence, and wherein the second L-DNA sequence is associated with the first detectable marker and a second zip-coded L-DNA analog probe complex that comprises a) a third segment that comprises a second PNA sequence, wherein the second PNA sequence will effectively hybridize to a second target sequence, and a third L-DNA sequence, the third L-DNA sequence being associated with the second PNA sequence, b) a second detectable marker, and c) a fourth segment that comprises a fourth zip-coded L-DNA sequence, the fourth zip-coded L-DNA sequence will effectively hybridize to said third L-DNA sequence, and the fourth L-DNA sequence is associated with the second detectable marker. In another embodiment, the sequence of said second and said fourth L-DNA sequences are different from one another.

In another aspect, a kit of zip-coded analog probe complexes is provided. The kit comprises a set of zip-coded L-DNA analog probe complexes described above and materials for performing an in situ hybridization.

In another aspect, a method of detecting co-localization of a first and a second target nucleic acid is provided. The method comprises contacting a first and a second zip-coded L-DNA analog probe complex with a nucleic acid and observing the localization of the zip-coded L-DNA analog probe complexes. The observation of both zip-coded L-DNA analog probe complexes in a single locale will demonstrate the co-localization of a first and a second target nucleic acid. In another embodiment, the zip-coded analog probe complexes have a first and a second detectable marker, and the observation of the localization of the zip-coded analog probe complexes is achieved by observing a signal from the detectable marker. In another embodiment, the detectable markers are quantum dots. In another embodiment, the first detectable marker is a red quantum dot and the second detectable marker is a blue quantum dot. In another embodiment, the first and the second target nucleic acid is mRNA. In another embodiment, the target nucleic acids are located within a neuron. In another embodiment, the observation of the co-localization of the first zip-coded analog probe complex and the second zip-coded analog probe complex are done in real time.

In another aspect, a hybrid detectable marker is provided. The hybrid detectable marker comprises a fluorescent moiety, a superparamagnetic moiety, and a substrate, which combines the fluorescent moiety with the superparamagnetic moiety. In one embodiment, the hybrid detectable marker further comprises a nucleotide segment, the segment has a first sequence of zip-coded L-DNA, the sequence will effectively hybridize to a second sequence of zip-coded L-DNA. In another embodiment, the hybrid detectable marker further comprises a second segment, the second segment comprises the second zip-coded L-DNA sequence, the second L-DNA sequence will effectively hybridize the second zip-coded L-DNA sequence and the first zip-coded L-DNA sequence. In another embodiment, the second segment further comprises a PNA sequence connected to the second zip-coded L-DNA sequence; the PNA sequence will hybridize to a target sequence. In another embodiment, the substrate is a polymer shell. In another embodiment, the polymer is polystyrene. In another embodiment, the superparamagnetic moiety is superparamagnetic iron oxide. In another embodiment, the fluorescent moiety is a Q dot. In another embodiment, the hybrid detectable marker further comprises a DNA sequence attached to the composite detectable marker, and a part of the DNA sequence can hybridize to a first target sequence. In another embodiment, the hybrid detectable marker further comprises an antibody, wherein the antibody can bond to an antigen while said antibody is attached to the composite detectable marker. In another embodiment, the superparamagnetic moiety is a core of the detectable marker, the fluorescent marker is a Q dot, and the substrate holding the superparamagnetic moiety and the fluorescent moiety together is a polymer shell of polystyrene, and the composite detectable marker further comprises a) a first segment comprising a first L-DNA sequence, said first segment being associated with the polystyrene shell and wherein said first L-DNA sequence will hybridize to a second L-DNA sequence, and b) a second segment comprising the second L-DNA sequence, wherein said second L-DNA sequence will hybridize to said first L-DNA sequence and wherein said second L-DNA sequence is connected to a PNA sequence, wherein said PNA sequence will hybridize to a target sequence.

In another aspect, a method of following a single composite detectable marker throughout a host to a host cell is provided. The method comprises administering a hybrid detectable marker described above to a host and a host organ, monitoring the movement of the hybrid detectable marker through the host and the host organ and tissue using a MRI device, and monitoring the movement of the composite detectable marker through a host cell through a fluorescence detection device, thereby following a single composite detectable marker throughout a host to a host cell.

In another aspect, a method of in-situ hybridization of a sample is provided. The method comprises contacting an analog probe complex with a sample comprising a target sequence, the analog probe complex comprises a) a first segment that comprises a first PNA sequence, the first PNA sequence will hybridize to a target sequence, and b) a second segment, the second segment that comprises a first L-DNA sequence, wherein the first L-DNA sequence can dissociably bind to the first PNA sequence, c) a fluorophore attached to the first segment, d) a quencher attached to the second segment, allowing the PNA sequence to bind to the target sequence, fixing the sample, and observing a fluorescence from the fluorophore. In another embodiment, the PNA sequence is longer than the L-DNA sequence. In one embodiment, the target sequence is mRNA. In another embodiment, no wash step is performed after contacting an analog probe and after observing a fluoresecence. In another embodiment, a temperature of a mixture in which the contacting step occurs is lower than a temperature of a mixture that would be required for the PNA segment from an otherwise identical PNA/D-DNA analog probe complex to dissociate in the presence of a target. In another embodiment, the fixing of the sample occurs before the analog probe complex is contacted with the sample.

In another aspect, a kit for wash free in situ hybridization is provided. The kit comprises an analog probe complex that comprises 1) a first section comprising a fluorescent marker and a PNA sequence, wherein the PNA sequence will hybridize to a target sequence and can dissociably bind to a L-DNA sequence, and 2) a second section comprising a L-DNA sequence and a quencher, wherein the L-DNA sequence can dissociably bind to the PNA sequence, and a fixation reagent. In another embodiment, the kit further comprises a low ionic strength solution. The low ionic strength is sufficient for dissociation of a L-DNA/PNA hybridized probe, but insufficient for effective dissociation of a D-DNA/PNA probe. In another embodiment, the fluorescent marker is a quantum dot.

In one aspect, an analog probe complex for the detection of nucleic acids is provided. The analog probe complex comprises a first nucleic acid analog segment, the segment comprises a means to associate with a target sequence, and a second nucleic acid analog segment, the second segment comprises a means to dissociably associate to the first nucleic acid analog segment. The dissociable association is dependent upon an association of the first segment to a target sequence.

In one aspect, an analog probe complex for the detection of nucleic acids is provided. The analog probe complex comprises a first nucleic acid analog segment that comprises a means for detecting a target sequence, a second nucleic acid analog segment that comprises a means for detecting the second nucleic acid analog segment, and a means for hybridizing the first segment with the second segment.

In one aspect, a hybrid detectable marker is provided. The hybrid detectable marker comprises a first detectable marker for detection, wherein the first detectable marker is detectable through a first method, a second detectable marker for detection, wherein said second detectable marker is detectable through a second method, a means for associating the first and second detectable markers, and a means for connecting said first and second detectable markers to a probe sequence.

These and other features of the present teachings are set forth herein.

### BRIEF DESCRIPTION OF THE DRAWINGS

One of ordinary skill in the art will understand that the drawings, described below, are for illustration purposes only. The drawings are not intended to limit the scope of the present teachings in any way.

FIG. 1 is a drawing of a traditional molecular beacon, free and bound to a target sequence.

FIG. 2 is a structural depiction of a PNA sequence compared to a DNA sequence.

FIG. 3 is a structural depiction of natural D-DNA (left) compared to the L-DNA analog (right).

FIG. 4A is a drawing of an analog probe complex, hybridized together (left) and hybridized to a target sequence (right).

FIG. 4B is a drawing of an example of an analog probe complex being used in an amplification protocol, such as a TAQMAN® protocol.

FIG. 4C is a drawing of an example of an analog probe complex with a metal nanoparticle or a quantum dot for a quencher which is being used in a protocol that is similar to that shown in FIG. 4B.

FIG. 5 is a drawing of a different embodiment of a set of two analog probe complexes, hybridized to a target sequence.

FIG. 6 is a drawing of a hybrid detectable marker (DM).

FIG. 7 is a drawing of another embodiment of an analog probe complex.

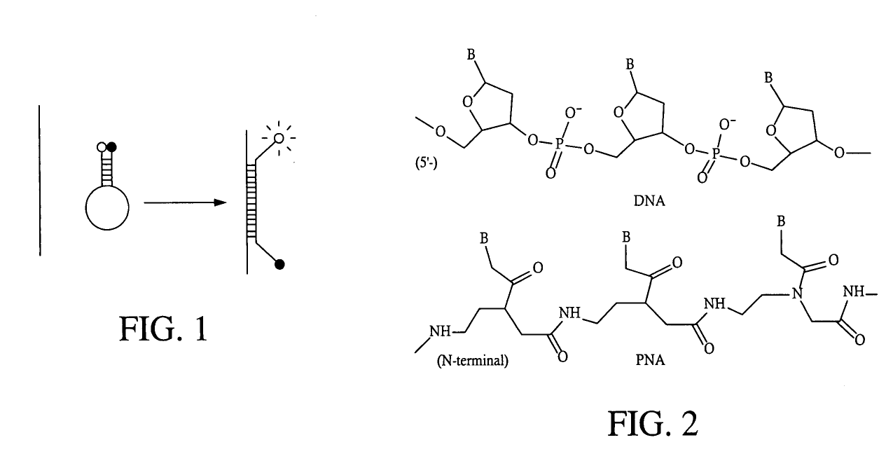

### DETAILED DESCRIPTION OF VARIOUS EMBODIMENTS

It has been discovered that L-DNA is a useful nucleotide in the formation of various analog probe complexes for the detection of particular target sequences. Since L-DNA does not, by itself, hybridize to the naturally occurring form of DNA (D-DNA) or RNA, L-DNA is useful in situations where binding of the DNA segment to a target sequence is not desirable or required. For example, a L-DNA segment can be useful as a leaving group that results in a detectable change in fluorescence. Additionally, since L-DNA is not a natural substrate for many enzymes, the stability of a L-DNA probe can be greater than for a D-DNA probe. Several embodiments employing L-DNA are contemplated. In one embodiment, the combination of a L-DNA segment and a PNA segment results in a completely artificial probe complex that is highly stable and highly efficient.

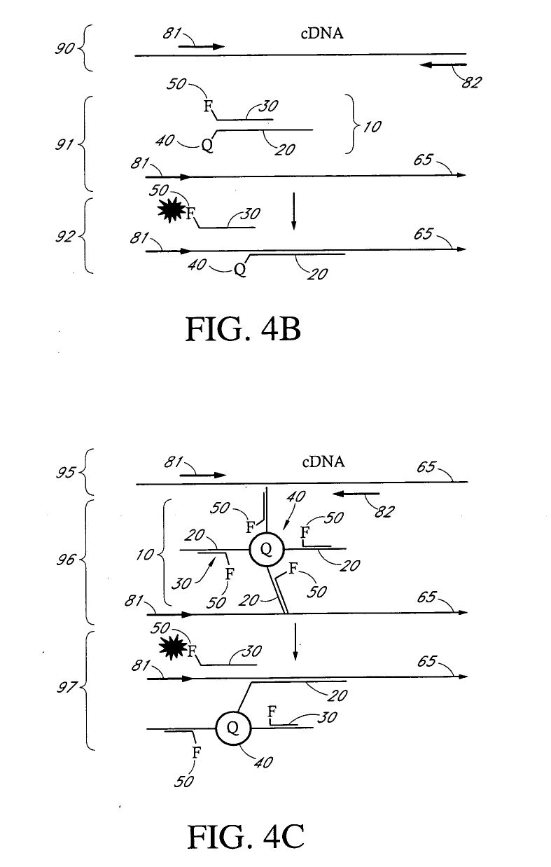

In one embodiment, a L-DNA segment is combined, through hybridization, with a PNA segment to fashion a novel self-indicating analog probe complex, for example, as shown in FIG. 4A. The PNA segment can hybridize to both a target sequence and a sequence on the L-DNA segment. Additionally, the L-DNA segment is designed to hybridize to the PNA segment, in a similar location to where the PNA segment hybridizes to the target sequence; thus, resulting in the separation of the L-DNA segment and the PNA segment when the PNA segment binds to a target sequence.

The PNA segment can have a fluorescent marker attached to one end and the L-DNA segment can have a fluorescent modulator or quencher attached to one end. Since the two segments contain complementary sequences, they will typically be hybridized together. This results in the quencher being placed in close proximity to the fluorescent marker when the probe is isolated and stably hybridized. However, as described, the presence of a target sequence will result in the L-DNA sequence being removed, the separation of the two strands, and the separation of the fluorescent marker and the quencher. This separation of fluorescent marker and modulator will result in a change in fluorescent signal, which is detectable. Thus, the binding of this probe complex, comprising analog nucleic acids, will result in a signal being generated. For example, FRET may be used to observe when the L-DNA probe and the PNA probe dissociate upon binding of the PNA probe to a target sequence. Alternatively, other fluorescence techniques or nonfluorescent techniques can be employed for detection.

The analog probe complexes described herein have several advantages over traditional probes. For example, since neither PNA nor L-DNA is native to cells, their risk of degradation is greatly reduced, since the cell's enzymes are designed for D-DNA manipulation. Additionally, since L-DNA will not bind to D-DNA, non-probe (or “probe complement”) binding is reduced if not eliminated. It was discovered that L-DNA does bind to PNA (an achiral compound) despite the fact that PNA is highly specific for particular sequences of D-DNA.

Additionally, the combined PNA/L-DNA analog probe complex can exhibit characteristics that allow for PNA to dissociate to form a PNA/D-RNA, or PNA/D-DNA, hybrid, but do not allow for that hybrid to be reversed into a PNA/L-DNA hybrid. For example, it was discovered that PNA will assume the same turn of a helix as the nucleotide segment to which it is bound. Thus, when PNA is bound to a L-DNA sequence, it takes on the three dimensional shapes associated with L-DNA. Likewise, when PNA is associated with D-RNA or D-DNA, the PNA segment will take on the opposite three dimensional shape, as compared to when it is bound to L-DNA. By making the PNA, or probe segment, longer than the L-DNA or blocker segment, the PNA segment will assume some structural elements similar to L-DNA, but it will retain additional free structural elements where it is not bound to the L-DNA. This part of the PNA segment may freely bind to a D-RNA or D-DNA target sequence. Additionally, since this segment binds to the D-DNA sequence, the L-DNA blocker sequence is removed from the rest of the PNA probe. Without being limited by any particular theory, it is believed that this reaction is assisted for two reasons. First, the change in orientation of the PNA segment starts a chain reaction of breaking bonds beyond where the PNA immediately binds to the D-RNA or D-DNA, perhaps by twisting the PNA backbone in the opposite direction. Additionally, it has been discovered that the D-RNA/PNA hybrid is more stable compared to the L-DNA/PNA hybrid. Thus, due to this higher stability and to the differences in size of the segments, one is able to create an analog probe complex that is effectively highly sensitive and specific, and has a very low background noise or rate of reversal of the hybridization.

Alternative labels can be used on the analog probe complex. For example, MRI agents and MRI blockers are used, thus allowing in vivo tracking of particular nucleotides on a much larger scale than simply on the cellular level.

Methods of using the PNA/L-DNA self-indicating analog probe complexes are also disclosed. In one embodiment, the detectable markers on the PNA and L-DNA segments are fluorescent in nature, allowing visualization of the presence of target nucleic acids via the observation of fluorescence. In another embodiment, the detectable marker is detectable through the use of a MRI or similar instrument, allowing for the tracking of the target sequence throughout a body or tissue.

The analog probe complexes can be used for performing homogeneous in-situ hybridization (HISH). Thus, in one embodiment, the analog probe complex is administered to a sample, allowed to hybridize to targets on the sample and then the probe is observed without first washing the sample free of the remaining analog probe complex. These can be any PNA probe complex, preferably one with a high signal to noise ratio. Thus, PNA/LNA, PNA/D-DNA, or other PNA/nucleotide analog probe complexes can also be used.

Kits are also provided comprising a PNA segment with a detectable marker that hybridizes to a target sequence and a L-DNA segment. The kit can also comprise both a PNA and a L-DNA segment, but the actual target binding sequence (probe sequence) is added by the user of the kit.

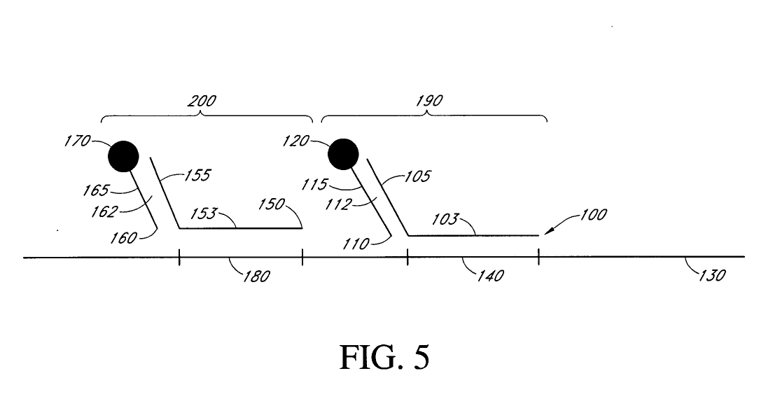

In one embodiment a zip-coded analog probe complex is provided, for example as shown in FIG. 5. In one example, the zip-coded analog probe complex comprises a L-DNA/PNA composition and it allows for the ready attachment of PNA sequences to other compositions, such as fluorescent detectable markers (DMs). This can be achieved through the use of zip-coded sections of complementary L-DNA segments. One of the zip-coded L-DNA sequences is attached to a PNA sequence; the PNA segment or sequence will hybridize to a target sequence. Another zip-coded L-DNA segment that is complementary to the first zip-coded L-DNA sequence is attached to a detectable marker. By allowing the two L-DNA zip-coded sequences to hybridize, one allows the attachment of a probe comprising sequence to any nucleic acid sequence. Thus, different DMs can be added to different PNA segments simply by hybridization of the two zip-coded sequences. One advantage of such an analog probe complex is that since the sequences are L-DNA sequences, they are very unlikely to bind to other D-DNA, D-RNA, or other naturally occurring nucleotide sequences in an in vivo system. Additionally, they can be more resistant to degradation, as discussed herein. Other embodiments of zip-coded probes and their uses are also discussed in U.S. Patent Application No. 60/584,799, filed Jun. 30, 2004, hereby incorporated by reference in its entirety.

In some embodiments, a set of zip-coded L-DNA/PNA analog probe complexes are provided, each probe having a different probing sequence to detect a different target sequence, such as an mRNA target sequence, and each probe complex having a different DM.

In some embodiments, methods are taught for determining the co-target localization of target sequences. One embodiment of such a method involves the use of two zip-coded L-DNA/PNA analog probe complexes, one which binds to a first target sequence and has a first detectable marker, and a second one that binds to a second target sequence and has a second detectable marker. By observing the presence of both markers simultaneously, and if the markers move simultaneously, one will be able to determine if two target sequences are co-localized. These zip-coded probes or chimeric probes can be used in vivo for hybridization studies.

In another embodiment, a target sequence is monitored by a set of these probes without washing away the added probe complexes. For example, two different target sequences within the target nucleotide sequence are used to attach two zip-coded analog probe complexes, each complex with a different detectable marker (DM). While additional zip-coded analog probe complexes may still be free in solution, only those areas with both signals will indicate the presence of the target segment. Here, the method allows one to not only make sure that the two sequences are co-localized, but by observing only those areas with a combination of both probes, it also allows one to effectively follow the target sequence without having to wash away any excess probe complex.

In one embodiment, a hybrid detectable marker (DM) is taught. The DM allows for the localization of a target sequence on the cellular level and across the organism as well. The detectable marker can comprise a superparamagnetic core and a selection of fluorescent markers, Q-dots for example. The two can be held together with a polymer coating or shell. The DM can also be connected to various probe sequences, PNA sequences for example, through the use of a L-DNA-zip-coded section.

Definitions:

The term “configuration” refers to the spatial array of atoms that distinguishes stereoisomers (isomers of the same constitution) other than distinctions due to differences in conformation. Configurational isomers are stereoisomers that differ in configuration. Absolute configurations of the novel compositions herein are defined by their particular chiral centers (e.g., sugar carbon atoms). The chiral carbons are designated by means of alphabetic symbols for rotation: R for rectus and S for sinister, defined by the bond priority rules of Cahn, Ingold, and Prelog (“Organic Chemistry”, Fifth Edition, J. McMurry, Brooks/Cole, Pacific Grove, Calif., pp. 315-319 (2000)), unless otherwise specified. In one embodiment, enantiomeric isomers of DNA are contemplated. In one embodiment, enantiomeric isomers of RNA are contemplated. In one embodiment, enantiomeric isomers of any nucleotide or nucleobase are contemplated. Here, the configurational differences between the chiral carbons for normal DNA and analog DNA may be indicated by indicators such as “D-DNA” and “L-DNA,” which still refer to chirality of the molecule and which are defined further below.

The term “chimeric configurational” refers to a compound with covalently connected subunits comprising different stereochemical configurations.

“Nucleobase” means any nitrogen-containing heterocyclic moiety capable of forming Watson-Crick hydrogen bonds in pairing with a complementary nucleobase or nucleobase analog, e.g. a purine, a 7-deazapurine, or a pyrimidine. Typical nucleobases are the naturally occurring nucleobases adenine, guanine, cytosine, uracil, thymine, and analogs (Seela, U.S. Pat. No. 5,446,139) of the naturally occurring nucleobases, e.g. 7-deazaadenine, 7-deazaguanine, 7-deaza-8-azaguanine, 7-deaza-8-azaadenine, inosine, nebularine, nitropyrrole (Bergstrom, J. Amer. Chem. Soc. 117:1201-09 (1995)), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman, in Practical Handbook of Biochemistry and Molecular Biology, pp. 385-394, CRC Press, Boca Raton, Fla. (1989)). The term “nucleobase” shall include those naturally occurring and those non-naturally occurring heterocyclic moieties commonly known to those who utilize nucleic acid technology or utilize peptide nucleic acid technology to thereby generate polymers which can sequence specifically bind to nucleic acids.

“Nucleoside” refers to a compound consisting of a nucleobase linked to the C-1′ carbon of a sugar, such as ribose, arabinose, xylose, and pyranose, in the natural .beta. or the alpha. anomeric configuration. The sugar can be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g. 2′-O-methyl, 4′-alpha-anomeric nucleotides, 1′-alpha-anomeric nucleotides (Asseline Nucl. Acids Res. 19:4067-74 (1991)), 2′-4′- and 3′-4′-linked and other “locked” or “LNA”, bicyclic sugar modifications (WO 98/22489; WO 98/39352; WO 99/14226). Exemplary LNA sugar analogs within a polynucleotide include the structures on page 4 of U.S. Patent Publication 2003/0198980, published to Greenfield et al., on Oct. 23, 2003, where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi Nucl. Acids Res. 21:4159-65 (1993); Fujimori, J. Amer. Chem. Soc. 112:7435 (1990); Urata, Nucleic Acids Symposium Ser. No. 29:69-70 (1993)). When the nucleobase is purine, e.g. A or G, the ribose sugar is usually attached to the N⁹-position of the nucleobase. When the nucleobase is pyrimidine, e.g. C, T or U, the pentose sugar is usually attached to the N′-position of the nucleobase (Kornberg and Baker, DNA Replication, 2^{nd} Ed., Freeman, San Francisco, Calif. (1992)).

“Nucleotide” refers to a phosphate ester of a nucleoside, as a monomer unit or within a nucleic acid. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g..alpha.-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The term “nucleic acid” refers to natural, artificial or analog of nucleic acids, or combinations thereof.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted.

As used herein, the term “nucleobase sequence” is any section of a polymer which comprises nucleobase containing subunits. Non-limiting examples of suitable polymers or polymer segments include oligonucleotides, oligoribonucleotides, peptide nucleic acids and analogs or chimeras thereof.

“Sequence” as compared to “segment.” While the terms may be used interchangeably in some circumstances, the term “segment” is generally meant to denote an entire physical piece of a nucleobase sequence or polynucleotide sequence, although individual pieces or segments can be ligated together. A “sequence” is merely meant to denote those nucleobases or nucleotides that are required for a given function. Thus, a segment can have many sequences within it, meaning that it is one continuous chain with difference sequences with many possible functions. In comparison, a single sequence will normally only be one, or part of, a single segment. For example, in one embodiment, given a target sequence of CCATTACC, a probe segment with the sequence GGTAATGG, and a complementary probe sequence (i.e., probe complement), of TACC, the probe segment will comprise at least two sequences, one that will hybridize to the target (CCATTACC) and one that will hybridize to the complementary probe sequence (GGTA), although probably not simultaneously for any substantial period of time. In general, a “section” will include all parts connected in a sufficiently stable manner, for example, a nonhybridized manner. Examples of sections include items 41 and 51 in FIG. 4A.

An “analog” nucleic acid is a nucleic acid that is not normally found in a host to which it is being added. This includes an artificial or synthetic nucleic acid. Thus, for example, in one embodiment, PNA is an analog nucleic acid, as is L-DNA and LNA (locked nucleic acids), iso-C/iso-G, L-RNA, O-methyl RNA, or other analogs. In one embodiment, any modified nucleic will be encompassed within the term analog nucleic acid. In another embodiment an analog nucleic acid can be a nucleic acid that will not substantially hybridize to native nucleic acids in a system, but will hybridize to other analog nucleic acids; thus, PNA would not be an analog nucleic acid, but L-DNA would be an analog nucleic acid. In an embodiment, all that is meant by analog is that while the molecule will hybridize with another nucleic acid or nucleic acid analog, it is not treated as a nucleic acid by other enzymes or proteins. For example, while L-DNA can hybridize to PNA in an effective manner, L-DNA will not hybridize to D-DNA or D-RNA in a similar effective manner. Thus, nucleotides that can hybridize to a probe or target sequence, but lack at least one natural nucleotide characteristic, such as susceptibility to degradation by nucleases or binding to D-DNA or D-RNA, are analog nucleotides in some embodiments. Of course, the analog nucleotide need not have every difference.

In some circumstances, not all of a segment needs to be of an analog nucleic acid in order for the segment to qualify as such. In one embodiment, only enough of the segment or sequence is a nucleic acid analog so as to confer the desired properties of the nucleic acid analog onto the segment to which it is attached. Thus, for example, greater than 0% of each segment or sequence will be of a nucleic acid analog. For example, minimal to 1, 1-2, 2-5, 5-10, 10-20, 20-40, 40-60, 60-80, or 80-100 percent of the sequence or segment will be of an analog nucleic acid. In some embodiments, only nucleic acids that are immune from digestion from host nuclease enzymes will be considered analog nucleic acids. The analog nucleic acid or nucleotides need not be restricted to DNA forms alone. As stated above PNA forms are included, as well as other forms of modified or artificial RNAs, for example, L-RNA, O-methyl RNA, LNA or other artificial RNAs. The bases comprising the analog nucleic acids need not be altered and can be able to bind with an effective level of specificity to the probe sequence. Phosphate ester analogs are encompassed within the term analog, and they include: (i) C₁-C₄ alkylphosphonate, e.g., methylphosphonate; (ii) phosphoramidate; (iii) C₁-C₆ alkyl-phosphotriester; (iv) phosphorothioate; and (v) phosphorodithioate.

As used herein, the term “peptide nucleic acid” or “PNA” is any oligomer, linked polymer or chimeric oligomer, comprising two or more PNA subunits (residues), including any of the compounds referred to or claimed as peptide nucleic acids in U.S. Pat. No. 5,539,082, 5,527,675, 5,623,049, 5,714,331, 5,736,336, 5,773,571 or 5,786,571 (all of which are hereby incorporated by reference). The term “Peptide Nucleic Acid” or “PNA” shall also apply to those nucleic acid mimics described in the following publications: Diderichsen et al., Tett. Lett. 37:475-478 (1996); Fujii et al., Bioorg. Med. Chem. Lett. 7:637-627 (1997); Jordan et al., Bioorg. Med. Chem. Lett. 7:687-690 (1997); Krotz et al., Tett. Lett. 36:6941-6944 (1995); Lagriffoul et al., Bioorg. Med. Chem. Lett. 4:1081-1082 (1994); Lowe et al., J. Chem. Soc. Perkin Trans. 1, (1997) 1:539-546; Lowe et al., J. Chem. Soc. Perkin Trans. 11:547-554 (1997); Lowe et al., J. Chem. Soc. Perkin Trans. 11:555-560 (1997); and Petersen et al., Bioorg. Med. Chem. Lett. 6:793-796 (1996).

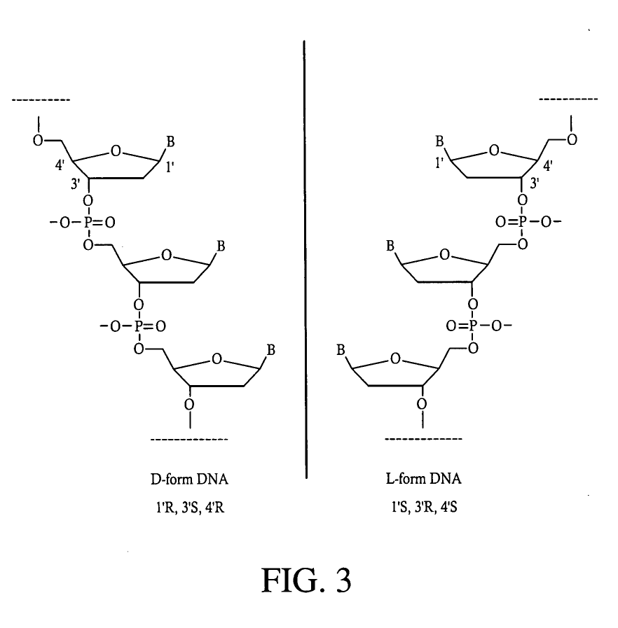

In one embodiment a PNA is a polymer comprising two or more PNA subunits of the formula 1 on page 6 of U.S. Patent Application 2003/0036059, to Coull et al., published Feb. 20, 2003. Each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms which can optionally contain a heteroatom or a substituted or unsubstituted aryl group. Each A is selected from the group consisting of a single bond, a group of the formula; —(CJ₂)ₛ— and a group of the formula; —(CJ₂)₅C(O)—, wherein, J is defined above and each s is an integer from one to five. The integer t is 1 or 2 and the integer u is 1 or 2. Each L is the same or different and is independently selected from the group consisting of J, adenine, cytosine, guanine, thymine, uridine, 5-methylcytosine, 2-aminopurine, 2-amino-6-chloropurine, 2,6-diaminopurine, hypoxanthine, pseudoisocytosine, 2-thiouracil, 2-thiothymidine, other naturally occurring nucleobase analogs, other non-naturally occurring nucleobases, substituted and unsubstituted aromatic moieties, biotin and fluorescein. In the most preferred embodiment, a PNA subunit consists of a naturally occurring or non-naturally occurring nucleobase attached to the aza nitrogen of the N-[2-(aminoethyl)]glycine backbone through a methylene carbonyl linkage. An example of a PNA polymer is shown in FIG. 2

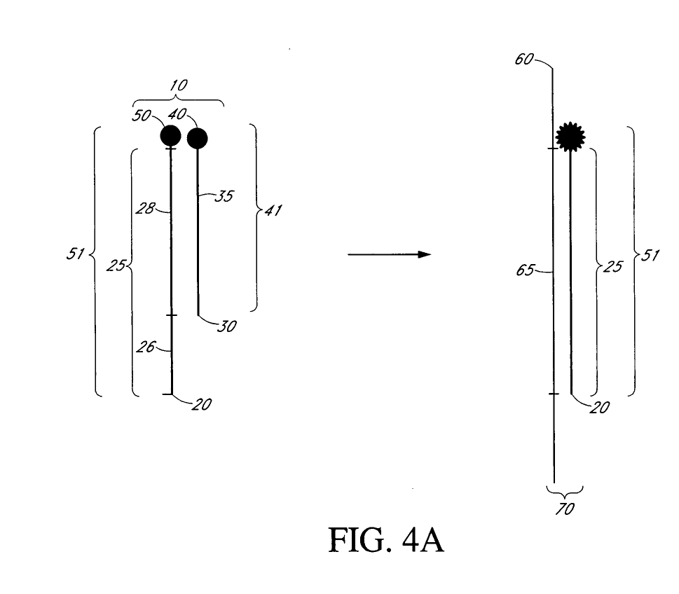

A L-DNA is a DNA whose three dimensional structure is different from D-DNA, the structure of D-DNA is shown in FIG. 3. In one embodiment, L-DNA comprises at least three structural differences as compared to D-DNA, as L-DNA is 1′S, 3′R, and 4′S. In another embodiment, L-DNA only has one or two differences as compared to D-DNA, for example 1′S, 3′R, 4′R; 1′R, 3′R, 4′S; or 1′R, 3′R, 4′. In another embodiment, the enantiomeric differences are present within the bases themselves. The L-DNA need not be an exact mirrored structure of D-DNA in any respect apart from at least one enantiomeric bond difference. In this embodiment, it is only relevant that the “L-DNA” binds to the probe sequence, or other similar nucleotide analog, and does not bind the sequence type of the target. In another embodiment, while the target sequence can bind to a probe sequence, there is no significant species present that can bind to the L-DNA segment. This helps to make certain that signaling from the analog probe complex comes from the detection of a target sequence.

The term “chimeric configurational oligonucleotide” means a continuous oligonucleotide comprising nucleotides of different configurations. The term “chimeric configurational nucleic acid” means a continuous nucleic acid sequence comprising nucleic acids of different configurations. Chimeric configurational oligonucleotides can have one or more portions of L-form nucleotides and one or more portions of D-form nucleotides. The entire nucleotide need not be in the opposite conformation. The chimeric configurational oligonucleotide can comprise additional types of oligonucleotides as well.

“Self-indicating” analog probe complexes are probe complexes where the binding of the probe sequence 25 to the target sequence 65 results in a signal, or indication, occurring from the probe complex, an example of which is shown in FIG. 4A. As described below, the signal may originate from any part of the probe complex or individual parts of the probe complex. This signal allows one to observe the presence of a target sequence in a sample. The signal or indication that occurs upon the detection of a sequence can be an increase in fluorescence of a donor fluorophore due to a decrease in a FRET interaction between the donor fluorophore on the probe segment 20 and the acceptor fluorophore on the complementary segment 30. Other such signaling events are discussed herein and they are not limited to fluorescence.

“Polypeptide” refers to a polymer including proteins, synthetic peptides, antibodies, peptide analogs, and peptidomimetics in which the monomers are amino acids and are joined together through amide bonds. When the amino acids are alpha-amino acids, either the L-optical isomer or the D-optical isomer can be used. Additionally, unnatural amino acids, for example, valanine, phenylglycine and homoarginine are also included. Commonly encountered amino acids that are not gene-encoded can also be used in the present invention. The amino acids used can be either the D- or L-optical isomer. In addition, other peptidomimetics can also be useful. For a general review, see Spatola, A. F., in Chemistry and Biochemistry of Amino Acids, Peptides and Proteins, B. Weinstein, eds., Marcel Dekker, New York, p. 267 (1983).

The term “amino acid” refers to naturally occurring and synthetic amino acids, as well as amino acid analogs that contain an amino group and a carboxylic acid group.

“Attachment site” refers to a site on a moiety or a molecule, e.g. a quencher, a fluorescent dye, or a polynucleotide, to which is covalently attached, or capable of being covalently attached, a linker or another moiety. In one embodiment, the attachment need only be sufficient for the use desired, and need not actually be covalent.

“Linker” refers to a chemical moiety in a molecule comprising a covalent bond or a chain of atoms that covalently attaches one moiety or molecule to another, e.g. a quencher to a polynucleotide. A “cleavable linker” is a linker that has one or more covalent bonds which can be broken by the result of a reaction or condition. For example, an ester in a molecule is a linker that can be cleaved by a reagent, e.g. sodium hydroxide, resulting in a carboxylate-containing fragment and a hydroxyl-containing product.

“Reactive linking group” refers to a chemically reactive substituent or moiety, e.g., a nucleophile or electrophile, on a molecule which is capable of reacting with another molecule to form a covalent bond. Reactive linking groups include active esters, which are commonly used for coupling with amine groups. For example, N-hydroxysuccinimide (NHS) esters have selectivity toward aliphatic amines to form aliphatic amide products which are very stable. Their reaction rate with aromatic amines, alcohols, phenols (tyrosine), and histidine is relatively low. Reaction of NHS esters with amines under nonaqueous conditions is facile, so they are useful for derivatization of small peptides and other low molecular weight biomolecules. Virtually any molecule that contains a carboxylic acid or that can be chemically modified to contain a carboxylic acid can be converted into its NHS ester. NHS esters are available with sulfonate groups that have improved water solubility.

“Substituted” as used herein refers to a molecule wherein one or more hydrogen atoms are replaced with one or more non-hydrogen atoms, functional groups or moieties. For example, an unsubstituted nitrogen is —NH₂, while a substituted nitrogen is —NHCH₃. Exemplary substituents include but are not limited to halo, e.g., fluorine and chlorine, C₁-C₈ alkyl, sulfate, sulfonate, sulfone, amino, ammonium, amido, nitrile, nitro, alkoxy (—OR where R is C₁-C₁₂ alkyl), phenoxy, aromatic, phenyl, polycyclic aromatic, heterocycle, water-solubilizing group, and linking moiety.

“Alkyl” means a saturated or unsaturated, branched, straight-chain, branched, cyclic, or substituted hydrocarbon radical derived by the removal of one hydrogen atom from a single carbon atom of a parent alkane, alkene, or alkyne. Typical alkyl groups consist of 1-12 saturated and/or unsaturated carbons, including, but not limited to, methyl, ethyl, cyanoethyl, isopropyl, butyl, and the like.

“Alkyldiyl” means a saturated or unsaturated, branched, straight chain, cyclic, or substituted hydrocarbon radical of 1-12 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl (—CH₂CH₂—), 1,3-propyldiyl (—CH₂CH₂CH₂—), 1,4-butyldiyl (—CH₂CH₂CH₂—CH₂—), and the like. “Alkoxydiyl” means an alkoxyl group having two monovalent radical centers derived by the removal of a hydrogen atom from the oxygen and a second radical derived by the removal of a hydrogen atom from a carbon atom. Typical alkoxydiyl radicals include, but are not limited to, methoxydiyl (—OCH₂—) and 1,2-ethoxydiyl or ethyleneoxy (—OCH₂CH₂—). “Alkylaminodiyl” means an alkylamino group having two monovalent radical centers derived by the removal of a hydrogen atom from the nitrogen and a second radical derived by the removal of a hydrogen atom from a carbon atom. Typical alkylaminodiyl radicals include, but are not limited to —NHCH₂—, —NHCH₂CH₂—, and —NHCH₂CH₂CH₂—. “Alkylamidediyl” means an alkylamide group having two monovalent radical centers derived by the removal of a hydrogen atom from the nitrogen and a second radical derived by the removal of a hydrogen atom from a carbon atom. Typical alkylamidediyl radicals include, but are not limited to —NHC(O)CH₂—, —NHC(O)CH₂CH₂—, and —NHC(O)CH₂CH₂CH₂—.

“Aryl” means a monovalent aromatic hydrocarbon radical of 5-14 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like, including substituted aryl groups.

“Aryldiyl” means an unsaturated cyclic or polycyclic hydrocarbon radical of 5-14 carbon atoms having a conjugated resonance electron system and at least two monovalent radical centers derived by the removal of two hydrogen atoms from two different carbon atoms of a parent aryl compound, including substituted aryldiyl groups.

“Substituted alkyl”, “substituted alkyldiyl”, “substituted aryl” and “substituted aryldiyl” mean alkyl, alkyldiyl, aryl and aryldiyl respectively, in which one or more hydrogen atoms are each independently replaced with another substituent. Typical substituents include, but are not limited to, F, Cl, Br, I, R, OH, —OR, —SR, SH, NH₂, NHR, NR₂, —⁺NR₃, —N—NR₂, —CX₃, —CN, —OCN, —SCN, —NCO, —NCS, —NO, —NO₂, —N₂+, —N₃, —NHC(O)R, —C(O)R, —C(O)NR₂—S(O)₂O—, —S(O)₂R, —OS(O)₂OR, —S(O)₂NR, —S(O)R, —OP(O)(OR)₂, —P(O)(OR)₂, —P(O)(O—)₂, —P(O)(OH)₂, —C(O)R, —C(O)X, —C(S)R, —C(O)OR, —CO₂—, —C(S)OR, —C(O)SR, —C(S)SR, —C(O)NR₂, —C(S)NR₂, —C(NR)NR₂, where each R is independently —H, C₁-C₆ alkyl, C₅-C₁₄ aryl, heterocycle, or linking group. Substituents also include divalent, bridging functionality, such as diazo (—N═N—), ester, ether, ketone, phosphate, alkyldiyl, and aryldiyl groups.

“Heterocycle” refers to a molecule with a ring system in which one or more ring atoms is a heteroatom, e.g. nitrogen, oxygen, and sulfur (as opposed to carbon).

“Enzymatically extendable” refers to a nucleotide which is: (i) capable of being enzymatically incorporated onto a terminus of a polynucleotide through the action of a polymerase enzyme, and (ii) capable of supporting further primer extension. Enzymatically extendable nucleotides include nucleotide 5′-triphosphates, i.e. dNTP and NTP, and labelled forms thereof.

“Enzymatically incorporatable” refers to a nucleotide which is capable of being enzymatically incorporated onto a terminus of a polynucleotide through the action of a polymerase enzyme. Enzymatically incorporatable nucleotides include dNTP, NTP, and 2′,3′-dideoxynucleotide 5′-triphosphates, i.e. ddNTP, and labelled forms thereof.

“Terminator nucleotide” means a nucleotide which is capable of being enzymatically incorporated onto a terminus of a polynucleotide through the action of a polymerase enzyme, but is then cannot be further extended, i.e. a terminator nucleotide is enzymatically incorporatable, but not enzymatically extendable. Examples of terminator nucleotides include ddNTP and 2′-deoxy, 3′-fluoro nucleotide 5′-triphosphates, and labelled forms thereof.

“Target”, “target polynucleotide”, “target sequence,” or similar terms mean a specific polynucleotide sequence, the presence or absence of which is to be detected, and that can be the subject of hybridization with a complementary polynucleotide, e.g. a primer or probe. The target sequence can be composed of DNA, RNA, analogs thereof, and including combinations thereof. The target can be single-stranded or double-stranded. In primer extension processes, the target polynucleotide which forms a hybridization duplex with the primer can also be referred to as a “template.” A template serves as a pattern for the synthesis of another, complementary nucleic acid (Concise Dictionary of Biomedicine and Molecular Biology, CPL Scientific Publishing Services, CRC Press, Newbury, UK (1996)). A target sequence can be derived from any living, or once living, organism, including but not limited to prokaryote, eukaryote, plant, animal, and virus. The target sequence can originate from a nucleus of a cell, e.g., genomic DNA, or can be extranuclear nucleic acid, e.g., plasmid, mitrochondrial nucleic acid, various RNAs, and the like. The target nucleic acid sequence can be first reverse-transcribed into cDNA if the target nucleic acid is RNA, if so desired. A variety of methods are available for obtaining a target nucleic acid sequence for use with the compositions and methods of the present invention. When the target sequence is obtained through isolation from a biological sample, possible isolation techniques include (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), or an automated DNA extractor (e.g., Model 341 DNA Extractor, Applied Biosystems, Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)). In one embodiment, the target sequence can be mRNA. In another embodiment the term “target sequence” can be any sequence of nucleobases in a polymer which is sought to be detected. The “target sequence” can comprise the entire polymer or can be a subsequence of the nucleobase polymer that is unique to the polymer of interest. Without limitation, the polymer comprising the “target sequence” can be a nucleic acid, a peptide nucleic acid, a chimera, a linked polymer, a conjugate or any other polymer comprising substituents (e.g. nucleobases) to which the PNA probe sequence can bind in a sequence specific manner. The target sequence can include any nature of nucleotide as well, for example, PNA, cDNA, mRNA, antisense RNA, siRNA, or microRNA (for a discussion of miRNA see Grishok et al., Cell, 106:2334 (2001); Carrington and Ambros, Science 301:336-338 (2003)).

The term “probe” means a polynucleotide that is capable of forming a duplex structure by hybridizing with a sequence of a target polynucleotide or sequence. Probes can be labelled, e.g., with a quencher moiety, or an energy transfer pair comprised of a fluorescent reporter and quencher.

The term “probe complement” or “complementary probe” sequence is generally meant to denote a sequence that is complementary to a sequence of a probe sequence. The probe complement need not bind to the entire probe sequence. In one embodiment, a probe complement is displaced by the binding of a part of the probe with a part of the target sequence. In another embodiment, the probe-target sequence hybridization is more stable than the probe-probe complement hybridization. This amount of increased stabilization can be any amount; for example, 1-5, 5-10, 10-20, 20-30, 30-40, 40-60, 60-80, 80-100, 100-150, 150-200, 200-300, 300-500, percent more stable or more, as determined through means known to one of skill in the art. The probe complement sequence can be 100% complementary to the relevant portion of the probe sequence. However, this number can be less, as long as they are substantially similar so that dissociation occurs significantly through binding of the probe sequence to the target sequence.

“Primer” means an oligonucleotide of defined sequence that is designed to hybridize with a complementary, primer-specific portion of a target sequence, a probe, or a ligation product, and undergo primer extension. A primer functions as the starting point for the polymerization of nucleotides (Concise Dictionary of Biomedicine and Molecular Biology, CPL Scientific Publishing Services, CRC Press, Newbury, UK (1996)).

The terms “duplex” means an intermolecular or intramolecular double-stranded portion of a nucleic acid which is base-paired through Watson-Crick, Hoogsteen, or other sequence-specific interactions of nucleobases. A duplex can consist of a primer and a template strand, or a probe and a target strand. A “hybrid” means a duplex, triplex, or other base-paired complex of nucleic acids interacting by base-specific interactions, e.g. hydrogen bonds.

The term “primer extension” means the process of elongating a primer that is annealed to a target in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand.

The term “label” refers to any moiety that can be associated with a polynucleotide and: (i) provide a detectable signal; (ii) interact with a second label to modify the detectable signal provided by the second label, e.g. FRET; (iii) stabilizes hybridization, e.g., duplex formation; (iv) confers a capture function, e.g., hydrophobic affinity, antibody/antigen, ionic complexation, or (v) change a physical property, such as electrophoretic mobility, hydrophobicity, hydrophilicity, solubility, or chromatographic behavior.

“Detectable marker”, “detection markers” “DM,” “detection moieties” or other similar terms refer to a type of label that allows for the observation of the label Detectable markers need not be visible through emission based methods. Thus, in one embodiment, a detectable marker is one that is detectable through more traditional means, such as antibody binding assays to the detectable marker. In other embodiments, the excitation, emission, or adsorption spectra of the detectable marker can be used for observation. Similarly, the marker modifier can be used to create changes in any of those spectra. In another embodiment, the detectable marker can create or inhibit some product that is itself detectable. In embodiments where the detectability of the detectable marker is modifiable to indicate the presence or absence of a L-DNA/PNA duplex (which in turn indicates the absence or presence of the target sequence), the detectable marker should still be modifiable in terms of its detection, although this can derive from something other than a marker modifier. For example, an antibody that only binds to the L-DNA/PNA hybridized duplex and not to the PNA/D-DNA hybridized duplex would allow the signal modification envisioned in these embodiments. In another embodiment, an antibody that bound only to free L-DNA sequences would also allow these probes to function appropriately. In such circumstances, the L-DNA segment itself can be considered the marker modifier and the antibody is the detectable marker. Thus, the detectable marker need not be covalently associated with the segments. In one embodiment, the absorption properties of the marker are what changes; thus, the detection is not of something emitted, but of something absorbed by the marker.

In one embodiment, the detectable marker emits a form of light or is sensitive to magnetic fields. The detectable marker can be a superparamagnetic nanoparticle or similar MRI detectable particle. The detectable marker can also be a fluorescent moiety. A fluorescent moiety is a moiety that fluoresces light, although the light need not be of the “visible” wavelength. A fluorescent marker is a compound that specifically emits light that can be detected. A fluorescent quencher is a label that alters the fluorescence of a fluorescent marker.

“Marker modifiers,” “MM” or other similar terms are compounds that allow for the detection of whether or not a PNA/L-DNA duplex is in a sample to be tested, or if the PNA and L-DNA segments have dissociated from each other, indicating the presence of a target sequence. The dissociation need not be complete. Anything that allows the observation of the probe sequence binding to a target sequence can be a marker modifier.

Marker modifiers (MMs) can be paired with DMs so that a first signal is generated by the DM; when the marker modifier and the DM are separated, a second signal is generated by the DM. Events which result in the pairing and separation of DMs and marker modifiers can thus be observed through changes in these signals. Thus, Beta-field shielders, when paired with a superparamagnetic DM, can be considered marker modifiers. Fluorescent modifiers or quenchers that alter the fluorescence characteristics of a fluorescent marker can also be a modifier. The marker modifier can also be a fluorescent probe that alters the fluorescence of the marker itself. The fluorescence modification need not be FRET based. The marker modifiers need not directly modify an emitted signal from a detectable marker. For example, an epitope and a change in an epitope between a PNA/L-DNA duplex and a free L-DNA segment or a PNA/D-DNA duplex can be sufficient to allow detection of the binding of the probe sequence to the target sequence. Thus, detection markers and marker modifiers can also be inherent in the complex itself.

As used herein, “energy transfer” refers to the process by which the excited state energy of an excited group, e.g. fluorescent reporter dye, is conveyed through space or through bonds to another group, e.g. a quencher moiety, which can attenuate (quench) or otherwise dissipate or transfer the energy. Energy transfer can occur through fluorescence resonance energy transfer, direct energy transfer, and other mechanisms, such as changes in the local environment of a marker (label) or changes in the mobility of the marker (label) itself. The exact energy transfer mechanisms is not limiting to the present embodiments. It is to be understood that any reference to energy transfer in the instant application encompasses all of these mechanistically-distinct phenomena.

“Energy transfer pair” refers to any two moieties that participate in energy transfer. Typically, one of the moieties acts as a fluorescent reporter, i.e. donor, and the other acts as a fluorescence quencher, i.e. acceptor (“Fluorescence resonance energy transfer.” Selvin P. (1995) Methods Enzymol 246:300-334; dos Remedios C. G. (1995) J. Struct. Biol. 115:175-185; “Resonance energy transfer: methods and applications.” Wu P. and Brand L. (1994) Anal Biochem 218:1-13). Fluorescence resonance energy transfer (FRET) is a distance-dependent interaction between two moieties in which excitation energy, i.e. light, is transferred from a donor (“reporter”) to an acceptor without emission of a photon. The acceptor can be fluorescent and emit the transferred energy at a longer wavelength, or it can be non-fluorescent and serve to diminish the detectable fluorescence of the reporter (quenching). FRET can be either an intermolecular or intramolecular event, and is dependent on the inverse sixth power of the separation of the donor and acceptor, making it useful over distances comparable with the dimensions of biological macromolecules. Thus, the spectral properties of the energy transfer pair as a whole change in some measurable way if the distance between the moieties is altered by some detectable amount. Self-quenching probes incorporating fluorescent donor-nonfluorescent acceptor combinations have been developed primarily for detection of proteolysis (Matayoshi, (1990) Science 247:954-958) and nucleic acid hybridization (“Detection of Energy Transfer and Fluorescence Quenching” Morrison, L., in Nonisotopic DNA Probe Techniques, L. Kricka, Ed., Academic Press, San Diego, (1992) pp. 311-352; Tyagi S. (1998) Nat. Biotechnol. 16:49-53; Tyagi S. (1996) Nat. Biotechnol 14:303-308). In most applications, the donor and acceptor dyes are different, in which case FRET can be detected by the appearance of sensitized fluorescence of the acceptor or by quenching of donor fluorescence.

The term “quenching” refers to a decrease in signal detectable moiety caused by a quencher moiety, regardless of the mechanism. For example, illumination of a fluorescent marker in the presence of a quencher leads to an emission signal that is less intense than expected, or even completely absent. The quencher can block 0-100% of the signal. For example, a quencher blocks 0-1,1-10, 10-20, 20-30, 30-40, 40-50, 50-60, 60-70, 70-80, 80-90, 90-95, 95-99, or 99-99.95, 99.95-99.98, 99.98-99.99, 99.99-100 percent of the signal of the detectable marker. In FRET based systems, quenching has it normal connotations. Shifts in fluorescent emission will also be considered as an adequate means for observing the changes in fluorescence from a detectable marker or the marker modifier. Thus, while the DM or the MM can emit just as much light, the emission of that light can be at different wavelengths than it was when the two segments were hybridized or separated. Thus, the change in signal between the hybridized analog probe complex and the separated analog probe complex need not be an absolute change in fluorescence intensity, since any change, e.g. absorption, emission spectra, intensity, that can be correlated to the two states of the probe can be sufficient for certain embodiments to function as desired.

The terms “annealing” and “hybridizing” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “solid support” refers to any solid phase material upon which an oligonucleotide is synthesized, attached or immobilized. Solid support encompasses terms such as “resin”, “solid phase”, and “support”. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled-pore-glass (CPG), or reverse-phase silica. The configuration of a solid support can be in the form of beads, spheres, particles, granules, a gel, or a surface. Surfaces can be planar, substantially planar, or non-planar. Solid supports can be porous or non-porous, and can have swelling or non-swelling characteristics. A solid support can be configured in the form of a well, depression or other container, vessel, feature or location. A plurality of solid supports can be configured in an array at various locations, addressable for robotic delivery of reagents, or by detection means including scanning by laser illumination and confocal or deflective light gathering.

“Array” or “microarray” means a predetermined spatial arrangement of polynucleotides present on a solid support or in an arrangement of vessels. Certain array formats are referred to as a “chip” or “biochip” (M. Schena, Ed. Microarray Biochip Technology, BioTechnique Books, Eaton Publishing, Natick, Mass. (2000)). An array can comprise a low-density number of addressable locations, e.g. 2 to about 12, medium-density, e.g. about a hundred or more locations, or a high-density number, e.g. a thousand or more. Typically, the array format is a geometrically-regular shape which allows for fabrication, handling, placement, stacking, reagent introduction, detection, and storage. The array can be configured in a row and column format, with regular spacing between each location. Alternatively, the locations can be bundled, mixed, or homogeneously blended for equalized treatment or sampling. An array can comprise a plurality of addressable locations configured so that each location is spatially addressable for high-throughput handling, robotic delivery, masking, or sampling of reagents, or by detection means including scanning by laser illumination and confocal or deflective light gathering.

The term “end-point analysis” refers to a method where data collection occurs only when a reaction is substantially complete.

The term “real-time analysis” refers to periodic monitoring during PCR. Certain systems such as the ABI 7700 and 7900HT Sequence Detection Systems (Applied Biosystems, Foster City, Calif.) conduct monitoring during each thermal cycle at a pre-determined or user-defined stage in each cycle. Real-time analysis of PCR with FRET probes measures fluorescent dye signal changes from cycle-to-cycle, preferably minus any internal control signals.

Analog Probe Complexes and Self Indicating Embodiments Thereof:

In one embodiment, two segments of analog nucleotide sequence are hybridized together to create an analog probe complex that, upon separation, can indicate the presence of a target sequence in a sample. In one embodiment, analog probe complexes indicate the presence of a target sequence through a detectable marker (“DM”) on one segment of the probe, and a marker modifier (“MM”) on a second segment of the probe. The two segments are initially hybridized to each other. Detection of the target sequence involves the separation of the two segments and thus the separation of the DM and the marker modifier, which in turn results in detectable change in signal from the DM. Since the hybridization of the probe to the target results in a change in a detectable signal, these embodiments of analog probe complexes can be termed “Self Indicating Analog Probe Complexes.” The analog probe complexes disclosed herein are made from functional nucleotide analogs, or artificial nucleotides.

One embodiment of such an analog probe complex 10 is shown in FIG. 4A. In this embodiment, the analog probe complex 10 comprises a first segment 20, which is the probe segment. The probe segment comprises a first sequence 28 that can bind to a sequence 35 on a second segment 30. The first segment 20 also comprises a second sequence 25 that can hybridize to a target sequence 65, which can be part of a third segment 60. The segment 20 and the sequences 25 and 28 can comprise PNA sequences. However, other analog nucleic acids can also be used, as appreciated by one of skill in the art, in light of the present teachings. The first segment 20 is also attached to a detectable marker 50 at one end of the segment. Additionally, the first segment 20 further comprises an additional sequence 26, which makes the length of the first segment 20 greater than the length of the second segment 30.

The second segment 30 comprises a complementary probe sequence 35 that is made of analog DNA, L-DNA for example. This analog DNA sequence or complementary probe sequence 35 is configured to dissociably hybridize to the first sequence 28 on the first segment 20, with dissociation occurring when the sequence 28 or 25 binds to a target sequence 65. Furthermore, the second segment 30 is attached to a marker modifier 40. As shown in FIG. 4A, the two segments 20 and 30 are hybridized, however, they need not be hybridized at all times. The sequence 35 on the second segment 30, functions as a complement to part of the probe sequence 28, requiring the at least partial dissociation of the sequence 35 in order for the sequence 28 to bind to a target sequence 65. The sequence 35 and the segment 30 are probe complementary sequences.

Each analog probe complex comprises two sections 51 and 41. The first section 51 will include a probe sequence 25 and a detectable marker 50, although the detectable marker can be inherent in the sequence itself. The second section 41 comprises a second segment 30 with a probe complement sequence 35 and a detectable marker 40, which again can be part of the sequence itself.

When the analog probe complex 10 is exposed to the target sequence 65, a strand exchange reaction occurs through branch migration, and the second segment 30, which can be L-DNA, for example, is replaced by the target sequence 65, resulting in the change of the fluorescence from the DM 50. In one embodiment, the reaction is made more favorable as the complementary sequence between PNA and target sequence, 65, which can be mRNA for example, is longer 26 than the complementary sequence between the PNA/L-DNA sequences 28 and 35, so that a more stable duplex 70 is formed.

In a FRET based example, the detectable marker 50 is a fluorophore, such as fluorescein (a donor) and marker modifier 40 is a quencher, such as tetramethylrhodamine (an acceptor). Thus, when the analog probe complex is a duplex 10, excitation at a fluorescein excitation wavelength will result in some degree of emission at the tetramethylrhodamine wavelength as the two labels 50 and 40 are close enough to allow a large amount of FRET to occur. However, very little fluorescein emission will occur. However, once the probe sequence 25 binds to the target sequence 65, the probe sequence 25, in particular probe sequence 28, and the complementary probe sequence 35 will have dehybridized, resulting in an increase in distance between the donor 50 and the acceptor 40. This in turn will result in a decrease in the amount of FRET between the two labels 50 and 40, which will result in a decrease in the amount of light emitted from the quencher and an increase in the amount of light emitted by the donor. Thus, this change will allow one to monitor the binding of the probe sequence 25 to the target sequence 65. As discussed herein, the interaction need not be FRET based as local changes in the environment of the probe can be sufficient to induce observable changes in the fluorescence of the DM 50.

Additionally, as will be appreciated by one of skill in the art, the actual placement of the DM 50 on the first segment 20 and the marker modifier 40 on the second segment 30, is not critical and the positions can be adjusted. They can be reversed or DMs 50 or MMs 40 can be placed on the target segment 60 as well, with predictable results in altered signal modifications upon hybridization. Additionally, the labels 50 and 40 need not be placed at the 5′ end of segment 20 and the 3′ end of segment 30. The positions of these labels 40 and 50 can be rearranged at opposite ends respectively, or with only one label at a different location. One of skill in the art will appreciate the fact that the degree of traditional FRET interaction can decrease with such placements, but as long as there is a detectable change, that will be sufficient for the operation of the complex. Additionally, not all of the labels 40 and 50 operate through traditional FRET interactions, as simple changes in the local environment of the probe can be sufficient or the first segment 20 or second segment 30 can be detected by other means, such as antibody binding, for example. In such cases, the placement of the labels 40 and 50 may not be as important, and in some situations, such as antibodies that selectively bind to an unhybridized structure of segment 30, not required at all.

The first segment of the probe 20 can comprise a PNA sequence. However, any material which can selectively hybridize to a target sequence, as well as hybridize to a second segment 30 with the characteristics that the second segment has, can be used. Additionally, the entire segment need not be made only of PNA. The percent of PNA can vary from minimal to 100%, for example, 1-10, 10-20, 20-30, 30-50, 50-70, 70-90, or more. The amount of PNA required can be enough to allow binding to the probe complement segment 30 and to provide selectivity to the probe and some amount of stability to the segment 20, compared to a segment that is made of D-DNA which can be degraded by enzymes. Alternatively, an amount of PNA, sufficient to grant the segment 20, any advantage described herein or known to one of skill in the art that PNA possesses over D-DNA, mRNA or other nucleotide sequence will be sufficient. The first segment 20 also comprises a probe sequence 25 and a sequence 28 that binds to the probe complement sequence 35.

The first segment 20 can also comprise an additional segment 26 which makes the first segment 20 longer than the second segment 30 by some amount. For example, 1 to any length, including 1, 2, 3, 4, 5, 6, 7, 8, 9, 10, 11, 12, 13-15, 15-18, or longer nucleotide units. This additional sequence 26, can optionally serve as additional sequence for the probe sequence 25 to allow for greater binding of the first segment 20 to the target sequence 65, as compared to the first segment 20 to the probe complement 30. This allows for a greater signal to noise ratio than would be achieved otherwise, as the process will favor the former duplex 70, rather than the later duplex 10.

In one embodiment, binding of the first segment 20 to D-DNA or mRNA can result in the reversal of the twist in the segment 20; this will encourage the second segment 30 to segregate from the duplex. Thus, complete binding of the probe sequence 25 need not be required for segregation and signaling to occur. The degree of binding of the probe sequence 25 to the target sequence 65 can be minimal and still cause dissociation. For example, a single nucleotide, or up to 100% of the sequence, including 1-5, 5-10, 10-20, 20-40, 40-60, 60-80, 80-100 percent of the sequence, 25 can bind to the target sequence 65 in order to have the second segment 25 dissociate from the first segment 20. The additional sequence 26 can be the first sequence to bind to the target sequence 65. Alternatively, the probe sequence 28 can be the first sequence to bind to the target sequence 65.

The second segment 30 comprises a sequence of analog nucleotides 35 which serves as a complementary sequence to the probe sequence 28. In one embodiment, these analog nucleotides can bind with a sequence 28 on the first segment 20 in a manner that is dependent upon the presence or absence of a target sequence 65. In one embodiment, the analog nucleotide sequence will not bind to a target sequence 65 sufficiently to interfere with the binding of the probe sequence 25. In one embodiment, the analog nucleotide sequence will relatively rapidly dissociate from the first segment 20 upon the initial binding of the probes sequence 25 to the target sequence 65. In one embodiment, the analog nucleotide sequence is relatively immune from cellular enzymes that traditionally break down native nucleotides (e.g., D-DNA). The sequence 35 can comprise 1′S, 3′R, 4′S DNA or “L-DNA.” In one embodiment, LNA (locked nucleic acid), L-RNA, or L-LNA or other analogs can also be used instead of L-DNA. As described above, the entire segment need not be made of the same analog nucleic acid, nor need the entire segment be made of analog nucleic acids. As described above, a percentage great enough to allow the desired advantages will be all that is required. The second segment can also comprise a marker modifier 40, or as discussed herein a DM 50, or simply be detectable to a DM which will selectively bind to the free from of the second segment 30 or sequence 35. Additional elements can be added to the second segment 30.

As used herein, the terms “substantially,” or “significantly” are general descriptors that indicate that insignificant changes in a particular element are permissible. Insignificant changes are those that do not prohibit function.

The analog probe complex 10 can comprise two separate segments 30 and 20; it can also comprise a single contiguous segment. The segment need not be connected via nucleotides. As will be appreciated by one of skill in the art, having the first and second segments connected can increase the probability that the second segment 30 will rehybridize to the first segment 20. Where the probe is to be used repeatedly, for example, this can be beneficial. The connection between the two segments can be of practically any length and of any material. As will be appreciated by one of skill in the art, the linker should not overly interfere with the binding of the first and second segments, nor with the other required functional aspects of the analog probe complex.

The target sequence 65 can be any nucleotide sequence. In one embodiment, the target sequence 65 is a natural nucleotide sequence such as mRNA or genomic DNA. The analog probe complexes can be used to determine concentrations of a whole host of important RNA species in tissue RNA isolates, for example tRNA, siRNA, viral RNA's, etc. Within a particular mRNA the concentrations of various exons after splicing could also be determined. RNA integrity could be monitored after storage by checking the concentrations of signature sequences. The target sequence can be in a cell, within a neuron, or within a living organism. The target sequence 65 can also be on an array or flowed across an array. The target sequence 65 can also be within an in situ experiment. The target sequence 65 can exist in a low copy number such that a probe with a high signal to noise ratio, such as the analog probe complex, is required. The target sequence 65 can also be part of a chemical reaction that is being monitored in real time, such as a PCR reaction, for example. For example, the target sequence can be combined with various components of a TAQMAN® protocol or other amplification protocol. The analog probe complexes can allow for the observation of real time linear amplification using RNA polymerase directly on the second strand synthesized cDNA without PCR or other cloned DNA fragments that contain a promoter sequences. The target sequence 65 can be an analog nucleotide sequence, such as L-DNA. As will be appreciated by one of skill in the art, this embodiment may not have all of the advantages as in the other embodiments; however, it will still be functional as an analog probe complex. As will be appreciated by one of skill in the art, and with the information provided herein, one of skill in the art will be able to apply many of these embodiments to practically any target with routine experimentation. Additionally, unlike traditional PNA probes, the analog probe complexes of some of these embodiments facilitates the delivery of these probes into live cells. For example, the partly hybridized PNA/L-DNA complex can be mixed with cationic lipid. The negative charges in the L-DNA phophodiester backbone will assist the PNA/L-DNA complex to associate with the cationic lipid. This mixture will be able to enter cells through an endocytotic pathway. In one embodiment, GPNA is used in the backbone to mimic a TAT sequence, as described in Peng Zhou et al. (JACS 125:6878-6879 (2003)).

As discussed below, the first segment 20 can also contain additional elements, such as a means to attach itself to a base or platform to allow arrays of analog probe complexes to be formed.

Additionally, as discussed herein, any combination of DM 50 and MM 40 can be used. For example MRI agents can be used to allow alternative detection techniques to be easily performed relatively deep inside of living organisms.

As discussed herein, detection of binding can vary depending upon the particular arrangement of detectable marker, 50 and marker modifier 40. Additionally, as discussed herein, the DM and MM need not be in every embodiment. While measuring changes in FRET interactions and changes in fluorescence due to changes in environment are relatively routine in the art, the detection of the binding event need not be limited to detection through the DM 50. In one embodiment, the detection of the binding event is through the detection of changes in fluorescent properties of the marker modifier 40 attached to the second segment. For example, a change in fluorescence characteristics, absorption for instance, can occur upon the separation of the second segment 30 from the first segment 20, thus allowing detection through changes in absorption or emission spectra or at single wavelengths. Alternatively, if the marker modifier 40 is also fluorescent, an antibody can be used to collect the dissociated second segment 30 and its concentration determined, thus allowing the amount of target sequence present to be determined.

The analog probe complexes disclosed herein have useful advantages over traditional nucleic acid probes. For example, nucleic acid constructs comprise a polynucleotide backbone whereas these analog probe complexes comprise a probing nucleobase sequence that is not completely a polynucleotide. Thus, where the analog probe complexes comprise PNA sequences, the PNA sequences will exhibit the favorable properties of PNA, such as, resistance to nuclease degradation, salt independent sequence hybridization to complementary nucleic acids and rapid hybridization kinetics.

Additionally, when the first segment and the second segment are not connected, it could allow for a better signal to noise ratio than in traditional molecular beacons. This occurs because, once a complementary sequence 35 has left the probe sequence 28, it is free to move into the bulk solution, decreasing the chances that it will bind again to the probe sequence. Furthermore, as the complementary probe sequence 35 is not a natural D-DNA sequence, the odds that the blocker sequence will interfere with a target sequence are greatly reduced. Additionally, as L-DNA, for example, is not a typical target of most cellular enzymes, the likelihood of having degradation of the second segment 30, and thus nonspecific signal from the analog probe complex is much lower than in any PNA/D-DNA molecular beacons.

Additionally, under normal conditions, L-DNA cannot be competed off the PNA by any complementary or partially complementary D-DNA or D-RNA that may coincidentally occur in the sample, cell or cell lysates under normal conditions. Only sequences complementary to the PNA will displace the L-DNA. Also, as mentioned above, L-DNA, once released from the PNA complement, will not normally interact with any polymerases or ligases or D-DNA binding proteins that are part of the protocols. In one embodiment, the analog probe complex has high sensitivity and low background noise; thus, it is possible to directly measure RNA targets without any amplification. In one embodiment, the fluorescent intensity in the analog probe complex has been quenched more than 99.5%, which makes it possible to be able to detect low copy numbers of RNAs.

In one embodiment, the probe sequence 25 target sequence 65 duplex 70 results in a stabilization of the target sequence. For example, the binding of PNA to mRNA can help to make the mRNA sequence less susceptible to native nucleases.

In another embodiment, the sets of PNA/L-DNA analog probe complexes are suitable for detecting or identifying the presence, absence or amount of two or more different target sequences which might be present in a sample. The characteristics of analog probe complexes suitable for the detection, identification or quantitation of target sequences have been previously described herein. The grouping of analog probe complexes can be grouped within sets characterized for specific detection of two or more target sequences.

Analog probe complex sets can comprise at least one PNA segment but need not comprise only PNA segments. For example, probe sets can comprise mixtures of PNA inert complex probes, other PNA probes and/or nucleic acid probes are also allowed, provided however that a set comprises at least one PNA inert complex probe as described herein. In some embodiments, at least one probe of the set is a complementary probe sequence 35.

Aspects of the present teachings may be further understood in light of the following examples, which should not be construed as limiting the scope of the present teachings in any way.

### EXAMPLE 1

This example demonstrates one method for making a self indicating analog probe complex. PNA will be synthesized using a modified DNA synthesizer. Alexa 680 (Molecular Probe), a fluorescent probe, will be linked to the N-terminal of the PNA and will be purified by reverse phase HPLC. The fluorophore Alexa is selected because of its long photobleaching lifetime and its long emission wavelength (705 nm) away from autofluorescence. The complementary L-DNA strand will be labelled at the 3′ end with “BLACK HOLE QUENCHER”™. The two segments will be allowed to hybridize together.

### EXAMPLE 2

This example demonstrates how the signal to noise ratio for the analog probe complex can be determined. Target sequence DNA will be added into a solution of a self indicating analog probe complex of Example 1. A fluorescence increase will be observed, which will indicate the hybridization of the analog probe complex with the target sequence. The S/N is estimated to be about 220.

### EXAMPLE 3

This example demonstrates one method by which the analog probe complex's resistance to both enzymatic degradation and protein binding can be determined. To a solution of self-indicating analog probe complexes, an amount of a DNA nuclease will be added: Following this addition, and at various periods of time thereafter, the fluorescence of the solution will be measured. This level of fluorescence will indicate the amount of binding and nucleotide degradation, with an increase in fluorescence indicating an increase in degradation. Following this, target sequence will be added to the solution to verify that other events have not either altered the DM or the analog probe complex. The addition of the target sequence will result in an increase in fluorescence, similar to that seen in Example 2.

Immobilization of an Analog Probe Complex to a Surface:

One or more analog probe complexes can be immobilized to a surface. The probe can be immobilized to the surface using the well known process of UV-crosslinking, for example. Alternatively, the first segment 20, a PNA oligomer, for example, is synthesized on the surface in a manner suitable for deprotection but not cleavage from the synthesis support.

The analog probe complex can be covalently linked to a surface by the reaction of a suitable functional groups on the probe and support. Functional groups such as amino groups, carboxylic acids and thiols can be incorporated in an analog probe complex by extension of one of the termini with suitable protected moieties (e.g. lysine, glutamic acid and cystine). When extending the terminus, one functional group of a branched amino acid such as lysine can be used to incorporate the donor or acceptor label at the appropriate position in the polymer (See: PNA Labeling) while the other functional group of the branch is used to optionally further extend the polymer and immobilize it to a surface.

Methods for the attachment of probes to surfaces generally involve the reaction of a nucleophilic group, (e.g. an amine or thiol) of the probe to be immobilized, with an electrophilic group on the support to be modified. Alternatively, the nucleophile can be present on the support and the electrophile (e.g. activated carboxylic acid) present on the analog probe complex. Because native PNA comprises an amino terminus, a PNA segment will not necessarily require modification to thereby immobilize it to a surface (See: Lester et al., Poster entitled “PNA Array Technology”). In one embodiment, only one segment, 20 or 30 will be attached to a surface. In situations where repeated use is preferred, both segments 20 and 30 can be attached or they can be linked.

Conditions suitable for the immobilization of a PNA sequence or segment to a surface will generally be similar to those conditions suitable for the labeling of a PNA (See discussion of PNA Labeling). The immobilization reaction is the equivalent of labeling the PNA whereby the label is substituted with the surface to which the PNA probe is to be covalently immobilized.

Numerous types of surfaces derivatized with amino groups, carboxylic acid groups, isocyantes, isothiocyanates and maleimide groups are commercially available. Non-limiting examples of suitable surfaces include membranes, glass, controlled pore glass, polystyrene particles (beads), silica and gold nanoparticles.

When immobilized to a surface, energy transfer between a DM 50 and a MM 40 will occur in the analog probe complex. Upon hybridization to a target sequence under suitable hybridization conditions, the location on the surface where the analog probe complex (of known sequence) is attached will generate detectable signal based on the measurable change in signal of at least one member of the immobilized analog probe complexes. Consequently, the intensity of the signal on the surface can be used to detect, identify or quantitate the presence or amount of a target sequence in a sample which contacts the surface to which the analog probe complex is immobilized. In one embodiment, detection of surface fluorescence will be used to detect hybridization to a target sequence.

As will be appreciated by one of skill in the art, and as discussed above, an example of a PNA segment 20 was particularly emphasized above, and in what follows. However, this is by way of convenience, as other analog nucleotides with similar characteristics can be used for the first segment 20 as well as the second segment 30, in some embodiments.

Detectable and Independently Detectable Moieties/Multiplex Analysis:

In some embodiments, a multiplex hybridization assay is performed. In a multiplex assay, numerous conditions of interest are simultaneously examined. Multiplex analysis relies on the ability to sort sample components or the data associated therewith, during or after the assay is completed. Distinct independently detectable moieties can be used to label the different inert probe complexes of a set. The ability to differentiate between and/or quantitate each of the independently detectable labels provides a means to multiplex a hybridization assay because the data which correlates with the hybridization of each of the distinctly (independently) labelled probe section 51 to a target sequence 65 can be correlated with the presence, absence or quantity of the target sequence 65 sought to be detected in a sample. Consequently, these multiplex assays can be used to simultaneously detect the presence, absence or amount of one or more target sequences 65 which can be present in the same sample in the same assay. Independently detectable fluorophores can be used as the detectable markers of a multiplex assay using probe segments 20. For example, two different probe sections 51 might be used to detect each of two different target sequences 65 wherein a fluorescein (green) labelled probe would be used to detect the first of the two target sequences and a rhodamine or Cy3 (red) labelled probe would be used to detect the second of the two target sequences. Consequently, a green, a red or a green and red signal in the assay would signify the presence of the first, second, and first and second target sequences, respectively. As will be appreciated by one of skill in the art, the probe section 51 can be used as part of an analog probe complex 10, so that the signal to noise ratio is much better. Thus, while the probe segment 20 is the segment that binds to the target sequence, it is contemplated that the entire analog probe complex 10, is initially used. However, in other embodiments, such as some of the zip-coded analog probe complexes, there may be no complementary segment that will dissociate from the probe complex. As will be appreciated by one of skill in the art, an entire probe section 51 need not necessarily be used, since just the probe segment 20 can be sufficient for some embodiments.

Arrays of Analog Probe Complexes:

Arrays are surfaces to which two or more probes of interest have been immobilized. In some embodiments, said immobilization occurs at predetermined locations. Arrays comprising both nucleic acid stereoisomer analog nucleic acids (such as L-DNA) and PNA probes have been described in the literature. The probe sequences immobilized to the array are chosen to interrogate a sample that can contain one or more target sequences of interest. Because the location and sequence of each probe is known, arrays are generally used to simultaneously detect, identify or quantitate the presence or amount of one or more target sequences in the sample. Thus, PNA arrays can be useful in diagnostic applications or in screening compounds for leads that might exhibit therapeutic utility.

For example, in a diagnostic assay a target sequence 65 is captured by the probe sequence 25 on the array surface and then the probe/target sequence complex 70 is detected using a secondary detection system. The probe/target sequence 70 complex can be detected using a second probe that hybridizes to another sequence of the target molecule of interest. Alternatively, a labelled antibody can be used to detect, identify or quantitate the presence of the probe/target sequence complex. Actual detection can be done through any number of devices, for example, a chemiluminescence analyzer can be used (such as the 1700 Chemiluminescent Microarray Analyzer from Applied Biosystems).

Since the composition of the analog probe complex is known because of its location on the surface of the array (e.g., because the analog probe complex was synthesized or attached to this position in the array), the composition of target sequence(s) can be directly detected, identified or quantitated by determining the location of detectable signal generated in the array. Because hybridization of the analog probe complex to a target sequence is self-indicating, no secondary detection system is needed to analyze the array for hybridization between the analog probe complex and the target sequence.

In some embodiments, arrays comprised of PNAs have the additional advantage that PNAs are highly stable and should not be degraded by enzymes which degrade nucleic acid. Therefore, PNA arrays should be reusable provided the nucleic acid from one sample can be stripped from the array prior to introduction of the second sample. Upon stripping of hybridized target sequences, signal on the array of analog probe complexes should again become reduced to background. Because PNAs are not degraded by heat or endonuclease and exonuclease activity, arrays of analog probe complexes should be suitable for simple and rapid regeneration by treatment with heat, nucleases or chemical denaturants such as aqueous solutions containing formamide, urea and/or sodium hydroxide. As described above, the segments 20 and 30 of such analog probe complexes 10 can be linked via a linker, or simply reassembled by rehybridization.

Methods

One method for the detection, identification or quantitation of a target sequence in a sample comprises contacting the sample with an analog probe complex 10 (as an example) and then detecting, identifying or quantitating the change in detectable signal associated with at least one DM 50 of an analog probe complex set. The signal for the correlation between detectable signal and hybridization is from the analog probe complex itself, e.g., it is self-indicating. This method is particularly well suited to analysis performed in a closed tube assay (that is, “homogeneous assays”). By closed tube assay it is meant that once the components of the assay have been combined, there is no need to open the tube or remove contents of the assay to determine the result. Since the tube need not be opened to determine the result, there will be some detectable or measurable change which occurs and which can be observed or quantitated without opening the tube or removing the contents of the assay. Thus, many closed tube assays rely on a change in fluorescence that can be observed with the eye or otherwise be detected and/or quantitated with a fluorescence instrument which uses the tube as the sample holder. Examples of such instruments include the Light Cycler (Idaho Technologies) and the Prism 7700 (PE Applied Biosystems). As will be appreciated by one of skill in the art, some of the analog probe complexes disclosed herein, while not self indicating, for example analog probe complex 190, can still be used in these methods and are considered as possible embodiments of these methods.

One example of a closed tube assay comprises the detection of nucleic acid target sequences that have been synthesized or amplified by operation of the assay. Non-limiting examples of nucleic acid synthesis or nucleic acid amplification reactions are Polymerase Chain Reaction (PCR), Ligase Chain Reaction (LCR), Strand Displacement Amplification (SDA), Transcription-Mediated Amplification (TMA), Ligase Detection Reaction (LDR), Rolling Circle Amplification (RCA) and Q-beta replicase. In one embodiment, the analog probe complexes present in the closed tube assay will generate detectable signal in response to target sequence production from the nucleic acid synthesis or nucleic acid amplification reaction occurring in the closed tube assay. The assay can also be an asymmetric PCR reaction.

### EXAMPLE 4

This example demonstrates how a self-indicating analog probe complex can be used for the direct determination of RNA sequence concentration without amplification. The level of fluorescence of a mixture of a saturating amount of an analog probe complex directed to a target RNA sequence and a sample containing the target RNA sequence will be determined by observing the amount of signal from a fluorescent marker, after the two have been mixed and enough time has passed to allow the probe section 51 to bind to the target sequence 65. Additional analog probe complex will be added until the increase or change in fluorescence is such as to indicate that the target sequence has been saturated with a probe section 51. Then, one will compare this degree of fluorescence with the results from when the same amount of analog probe complex is added to a target sequence of known concentrations. Thus, the concentration can be determined by determining the closest match between the sample tested and a series of controls.

### EXAMPLE 5

This example demonstrates one method for how the analog probe complexes described herein can be used for real-time monitoring of linear amplification by IVT (in vitro transcription) of RNA targets on Applied Biosystems International's SDS (Sequence Detection Systems). One will perform IVT (in vitro transcription) using T7 promoters and T7 RNA polymerase. One will reverse transcribe RNA using oligo dT primers tagged with T7 promoter sequences. Next, one will perform second strand synthesis to make double stranded template for IVT. Next one will perform IVT and monitor linear amplification with analog probe complex. As above, standards, or theoretical values can be used to determine the amount of RNA in a sample.

Other DNA dependent RNA polymerase systems, such as T3 RNA polymerase and SP6 RNA polymerase and their respective promoter sequences, can also be used in lieu of the T7 IVT system. For example, RT PCR RNA using an oligo dT primer tagged with T7 promoter sequences along with primers for the creation of double stranded cDNA. This will then be followed with IVT linear amplification as described above.

In some embodiments, the analog probe complexes can be used in an amplification protocol, such as a PCR protocol (e.g., a TAQMAN® type protocol). An example of such a use is demonstrated in FIG. 4B. FIG. 4B shows a PNA/L-DNA version of the analog probe complex 10 and one way that it could work in a TAQMAN® type or amplification protocol. In this embodiment, a quencher, or marker modifier, 40 is placed in close proximity to a fluorescing moiety, or detectable marker, 50 by having the quenching moiety 40 and the fluorescing moiety 50 on two segments 20 and 30 that have complementary sequences 35 and 28 (FIG. 4A). As above, the analog probe complex 10 works by displacement. The quencher 40 is separated from the fluorescing moiety 50 because one of the complementary segments 20 is longer than the other 30 and is complementary to the target polynucleotide 65 being monitored. The exposed part of the longer strand (FIG. 4A 26) hybridizes with the target polynucleotide and displaces the shorter segment 30 of the probe complex because the stability of the duplex 70 between the target and the longer probe strand is higher than that of the original quenched probe complex 10. In FIG. 4B the fluorescent moiety 50 is on the shorter segment 30; however, the positions of the fluorescent/quenching moieties can be reversed as changes in fluorescence are created by the separation of the two moieties and not necessarily their absolute positions. In one embodiment, the shorter segment 30 in FIG. 4B is made of L-DNA and the longer strand 20 is made of PNA. At least part of the PNA sequence should be complementary to the L-DNA sequence. However, in some embodiments, the segments or sequences of the probe or probe complex can include the D-configuration of DNA or other nucleotide analogues such as RNA, O-Methyl RNA, LNA, etc. as described herein.

FIG. 4B provides a further example of how an analog probe complex can be used to monitor the creation of an amplified product in real-time. One takes a target to be amplified, which includes a target sequence 65, and amplifies 90 it using a forward primer 81 and a reverse primer 82. Next, one adds 91 the analog probe complex 10 to the amplified sample, which contains the forward primer 81. Next, one allows 92 the analog probe complex to dissociate and for the longer segment 20 to bind to the target 65 in a sequence dependent manner. Thus, by measuring the increase in fluorescence due to the separation of the DM 50 and the MM 40, one can determine the amount of amplified target 65 that is present in the sample. As the analog probe complex can be relatively stable under relatively high temperatures or salt conditions, and as it dissociates primarily due to the presence of a sequence complementary to sequence 26, these probe complexes can have the advantage of being useful in conditions that might make other probe complexes nonspecifically dissociate and therefore useless.

Another probe complex that can be used in a real-time analysis is shown in FIG. 4C. As in FIG. 4B, one can first amplify 95 the target sequence 65. Next, one can add 96 the analog probe complex 10. In this embodiment, the analog probe complex 10 comprises multiple DMs 50 (e.g., fluorescent probes) associated with a single, relatively strong, quencher 40. In this particular embodiment, the MM 40 is a quencher and can be a silver, gold, or mercury nanoparticle, for example. Such relatively strong quenchers allow for multiple different fluorescing molecules to be used in the analog probe complex. After the analog probe complex is added to the amplified product, one can allow 97 the analog probe complex 10 to dissociate and the segment 20 to bind to the target sequence 65. As above, this will allow a change in fluorescence to occur. As will be appreciated by one of skill in the art, this particular combination can result in some of the fluorescent molecules remaining associated with the quencher. This can allow the complex to be repeatedly used, until all of the duplexes have been dissociated. Of course, this particular probe complex could be used in any of the other disclosed embodiments.

As will be appreciated by one of skill in the art, in some embodiments, the observation of the fluorescence or the change in fluorescence is done after the amplification is completed. In other embodiments, the observation of the fluorescence or the change in fluorescence is done while the amplification is still occurring or between cycles in the amplification process. Additionally, which segment is attached to the DM and which is attached to the MM can also be reversed.

In embodiments in which the analog probe complexes are to be used as fluorescent indicators in an amplification or TAQMAN® type protocol, the probe complexes can be used at approximately the same concentration as already employed for DNA or PNA molecular beacons or for TAQMAN® probes. Covalent tagging of PNA and L-DNA segments with DM and MM can be optimized structurally to minimize any residual fluorescent signal in the PNA/L-DNA complex. This can be done by moving the placement of the DM or MM across a given set of segments (20 and 30) until the largest change in fluorescence is observed upon dissociation of the two segments. The PNA segment can carry the fluorophore and the L-DNA the quencher, with the later in sufficient stoichiometric excess over the former that there are no PNA molecules free from L-DNA molecules except when the amplification process had generated enough target sequence to anneal to labeled PNA.

In some embodiments, the fluorescent signal can be measured at a temperature where the PNA/L-DNA complex is relatively unstable but the PNA-target duplex is stable. Attainment of a sufficiently broad thermal window to do this can be done by controlling the length differences between the two segments (e.g., the PNA segment and the L-DNA segment).

In some embodiments, the solvent in which the fluorescent measurement is made can contain supplemental material. For example, the solvent can contain an osmolyte, such as, betaine, sorbitol, glycerol or an organic co-solvent such as 2-pyrrolidinone or N-methylpyrrolidinone to assure that PNA that has dissociated from L-DNA remains sufficiently unfolded to react rapidly with the target sequence. Under these conditions, a more robust thermoresistant DNA polymerase than the Thermus enzymes can be desirable. For example, a hyperthermophilic DNA polymerase such as Vent or Vent (exo-). In other embodiments, the solvent lacks such additives.

In some embodiments, a kit or method is provided in which the above analog probe complex is used with Thermus DNA polymerases in a TAQMAN® like procedure. In other embodiments, a non-nick translating, hyperthermophilic, DNA polymerase with enhanced thermoresistance, the potential for proof-reading activity, and increased amplification fidelity is used. This can allow assays to be created regardless of the GC content.

In some embodiments, any device or method that uses TAQMAN® PCR signaling can be modified so as to use the analog probe complexes described herein as the product indicator.

### EXAMPLE 6

This example demonstrates one method by which one can test a probe for the detection of a target sequence, in particular, the presence of lacZ mRNA in an in vitro transcription system. The self indicating analog probe complex targeting the early coding region of the lacZ mRNA will be added into a mixture of all the components for an in vitro transcription system except the RNA polymerase. The fluorescence signal for the DM will be monitored. No dramatic signal increase will be observed from the DM until the addition of RNA polymerase. The signal will keep rising afterward, indicating that the production of mRNA is reflected by the hybridization of the PNA probe sequence.

### EXAMPLE 7

This example demonstrates one way that a self indicating analog probe complex can be used to monitor a target sequence in vivo. The same self-indicating analog probe complex from example 6 will be added to a “leaky strain” of E. coli that has been shown to be permeable to PNA (Sekiguchi et al., Proc Natl Acad Sci USA, 58(6):2315-20 (1967); Good et al., Curr Opin Biotechnol, 14(1):29-34 (2003)). Alternatively, a gentle single-cell electroporation method with carbon electrode can also be used. (Olofsson et al., Curr Opin Biotechnol, 2003, 14(1):29-34 (2003)). The fluorescence of the cells will be monitored. Increases in fluorescence will indicate an increased amount of target sequence. The level of fluorescence will be compared to volumes of solution similar in size to the cells with known amounts of self indicating probe and target sequence.

### EXAMPLE 8

This example demonstrates how one can use the analog probe complexes on human cells. The analog probe complex will be administered to the cells via microinjection. A tetracycline-inducible mammalian expression system (T-REX™) in human embryonic kidney 293 cell line (Yao et al., Hum Gene Ther, 9(13):1939-50 (1998)) will be used. A lacZ gene will be cloned under the control of Tet repressor. A plasmid encoding Tet repressor (pcDNA6/TR, from Invitrogen) will be cotransfected into 293 cell to establish the inducible lacZ gene expression system. With this system, the production of mRNA at basal and various induced levels will be possible. As described above, the analog probe complex will contain a probe sequence that binds to lacZ mRNA. Tracking of single mRNA molecules in three dimensions will be carried out with an Olympus confocal microscope and a scanning stage.

In Situ Hybridizations:

Because some embodiments of the analog probe complexes can be designed to be stable in the presence of enzymes found in cells, methods described herein can be particularly well suited to detecting a target sequence in a cell, tissue or organism, whether living or not. Thus, in-situ hybridization can be used as the assay format for detecting identifying or quantitating target organisms. This is especially relevant for the self indicating analog probe complexes 10 discussed above and the zip-coded analog probe complexes 190 discussed below. In situ hybridization (ISH) of cells and tissues can be used to determine the transcriptional profile of particular cells in different tissues and to detect the presence of various DNA configurations, for example duplicated genes in transformed cells. The current methods of labeling use radioactivity, enzymes or fluorescent dyes. Simply for clarification, any of the disclosed embodiments, whether they be hybridization dependent self-indicating analog probe complexes or zip-coded analog probe complexes can be employed for any of these methods, as will be appreciated by one of skill in the art. In particular, either can be useful for in situ hybridization compositions and methods.

Fluorescence in-situ hybridization (FISH or PNA-FISH) can also be used as the assay format. Exemplary methods for performing PNA-FISH can be found in: Thisted et al. Cell Vision, 3:358-363 (1996) or WIPO Patent Application WO97/18325, hereby incorporated by reference.

Target sequences in organisms that have been treated with analog probe complexes can be detected by several exemplary methods. The cells can be fixed on slides and then visualized with a microscope or laser scanning device. Alternatively, the cells can be fixed and then analyzed in a flow cytometer (See for example: Lansdorp et al.; WIPO Patent Application; WO97/14026). Slide scanners and flow cytometers are particularly useful for rapidly quantitating the number of target organisms present in a sample of interest.

Homogeneous In Situ Hybridization (HISH):

While the in situ hybridization method described above can be useful, the analog probe complexes can allow for additional modifications of an in-situ hybridization with some useful benefits. For example, regardless of the method of labeling, ISH (as discussed above) is a very complex procedure involving multiple complex steps that can take many days to complete. Long times are often required to complete the hybridization process and multiple wash steps are needed to remove background label. For ISH that detects RNA, the fixation process and the complexity of the procedure can be crucial to the availability of RNA for hybridization. This results from two events in the hybridization process. First, the RNA can be present but unavailable for reaction because it is trapped in cross-linked protein globs as a result of the fixation process. Second, even though hybridization can take place in the presence of RNAase inhibitors, long procedures risk the eventual digestion of the RNA target molecule. Any improvement in probe and label construction that could reduce hybridization time, improve hybridization levels and eliminate the necessity of having multiple washes to remove unreacted probe would be invaluable to this powerful tool.

In some embodiments, the analog probe complex can increase the rate of hybridization over nucleic acid probes and not require post hybridization washes. An embodiment of such a probe is shown in FIG. 4A. The probe can be designed to require only a hybridization step without washes, thus, this in situ hybridization procedure is denoted as a homogeneous in situ hybridization (HISH). The ability to detect the hybridization reaction without washes allows prefixation hybridization, which allows the detection of a target sequence, e.g., RNA, in protein granules unaffected by cross-linking fixatives or fixation procedures that precipitate protein (render the protein insoluble in an aqueous environment). Similarly, as discussed below, sets of zip-coded analog probe complexes, for example as shown in FIG. 5 can be used in a similar manner.

In one embodiment, another benefit of this novel probe design is that the hybridization reactions can be conducted under low ionic and low temperature conditions relative to nucleic acid/nucleic acid hybridizations. Low ionic conditions mean that target RNA secondary structures are destabilized and more available for hybridization with the probe. This means that the sensitivity of the reaction will be higher than those of comparable nucleic acid probes. Any reduction in ionic strength will represent an improvement. For example, a reduction of more than 0 to 1, 1-5, 5-10, 10-20, 20-50, 50-80, 80-90, 90-99, 99-100 percent in ionic strength will be an improvement. In one embodiment, the in situ is performed under physiological ionic strength conditions Additionally, lower temperatures decrease the rate of action of endogenous degrading enzymes when the in situ hybridization is conducted in a prefixation mode. Any decrease in temperature can be sufficient. For example, the temperature can be decreased by more than zero to 1, 1-5, 5-10, 10-20, 20-50, 50-80, 80-90, 90-99, 99-100 percent of the originally required temperature, where a 100% reduction will place the solution at 0° C.

Past probe structures did not permit homogeneous in situ hybridization due to their signal to noise ratio.

Homogeneous in-situ hybridization (HISH) can be based on a fluorescently self-indicating PNA/L-DNA analog probe complex. For example, one segment 20 of the probe 10 comprises PNA and carries a fluorescing moiety 50 on one end. This segment 20 also comprises a sequence 25 that is complementary to a specific internal sequence of one of the RNA or DNA members in the variety of RNA or DNA molecules being monitored. In this embodiment, the second segment 30 of the probe 10 comprises L-DNA nucleotides and contains a sequence 35 that is complementary to a sequence 28 in the first segment 20 that contains the fluorescing moiety, or detectable marker 50. Additionally, the second segment 30 carries a quenching moiety, or marker modifier 40 appropriately placed to quench the fluorescent moiety 50 on the first segment 20. RNA or DNA target molecules will hybridize with the first segment probe 20 at the probing sequence 25, and this will displace the L-DNA portion of the probe 30. Without intending to bound to any particular theory, this occurs because the PNA/RNA hybrid 70 is more stable than the PNA/L-DNA hybrid 10 and kinetically favored. The displacement of second segment 30 comprising L-DNA from the hybrid probe completely dissociates the quencher 40 from the fluorescent moiety 50. This is superior to traditional beacon probes that would rapidly reassociate should the target probe structure be disrupted thus requenching the signal.

In one embodiment, the fluorescent intensity of the analog probe complex 10 has been quenched by more than 99.5% when the probes are hybridized together. Thus the contribution of quenched unreacted probe is low enough to eliminate the necessity to include wash steps in the protocol.

In some embodiments, the use of PNA for the probe sequence has other advantages over the use of nucleic acids. PNA has an uncharged backbone and as a result does not have the same charge repulsion for the nucleic acid target as is the case for DNA or RNA probes. One of the results of this is that the rate of the hybridization reaction is greatly increased over DNA/DNA, DNA/RNA or RNA/RNA reactions. This means that hybridization times can be shortened and the levels of reaction are higher than those of conventional probes. For example, the time for the reaction can be shortened by as much as more than 0, to 1, 1-5, 5-10, 10-20, 20-40, 40-60, 60-80, 80-90, 90-95, 95-99, or greater percent of the time that it would have taken for a traditional probe.

In one embodiment, the use of a L-DNA complement 30 attached to a quencher 40 to quench the fluorescent moiety 50 on a PNA probe segment 20 has the advantage that the charges on the L-DNA segment 30 increase the solubility of the analog probe complex 10. L-DNA segment 30 is also insensitive to cellular enzymatic degradation increasing probe integrity. In addition, while the L-DNA sequence 35 will hybridize to the PNA complement 28 as efficiently as the correspondent, natural D-DNA, L-DNA will not hybridize to complementary D-DNA or RNA.

A PNA/L-DNA analog probe complex is not prone to enzymatic degradation in cellular environments, and is particularly suited for detecting specific mRNA in live cells. Without being limited by any particular theory, it is believed that the resistance to degradation can arise from the fact that the attacking of the nucleases on the DNA strand is blocked.

A PNA/LNA hybrid probe or other analog probe segment can be used to bind to the PNA probe segment that would have at least one similar characteristic as described above. PNA/D-DNA hybrid probe or other similar hybrid variant can be employed for the probe complex, as this need not be limited to complexes where both of the segments are analogs. The above and following discussion concerning analog probe complexes is by way of example only and is not intended to limit HISH to such analog probe complexes.

In one embodiment, the hybrid-PNA analog probe complex has a high signal to noise ratio compared to previous PNA and DNA beacons, permitting the high sensitivity necessary for detecting low copy numbers of genes in live cells. This is due to the effective quenching from the quencher on the complementary DNA, keeping the fluorescence background low. It is also superior to the DNA beacon due to the absence of sticky end pairing that connect among the DNA beacons. (Li et al., Anal Biochem, 312(2):251-4 (2003)). This signal to noise ratio can be increased by any amount more than zero. For example, 0 to 1, 1-2, 2-5, 5-10, 10-20, 20-30, 30-40, 40-50, 50-60, 60-70, 70-80, 80-90, 90-100, 100-140, 140-200, 200-250, 250-300, 300-400 percent or more, better than the equivalent DNA molecular beacon. In one embodiment the signal to noise ratio for an analog probe complex is over 200. The signal to noise can be 200-220, 220-240, 240-250, 250-270, 270-300, 300-350, 350-500, 500-750, 750-1000, or more.

In one embodiment, partly hybridized PNA/L-DNA is mixed with cationic lipid. The negative charges in the DNA phosphodiester backbone help the PNA-DNA hybrid to associate with the cationic lipid which can enter the cell through an endocytotic pathway. (Hamilton et al., Chem Biol, 6(6):343-51 (1999)). One of skill in the art will recognize that there are many alternative methods by which this may be done for the present embodiments.

While the above description of HISH featured PNA and L-DNA and a FRET based fluorescence detection system, as will be appreciated by one of skill in the art, in light of the teaching herein, any of these aspects can be readily manipulated to include other embodiments, e.g., such as various analog nucleotides or nonFRET fluorescence signaling or nonfluorescence signaling.

In one embodiment, it is an innate property of the one step in situ hybridization that the hydridization of the probe to the target RNA allows a change in the fluorescent signal. Therefore, the single hybridization step of this HISH procedure can obviate all the steps of other in situ hybridization procedures, for example, ISH, FISH, CISH. These steps generally include but are not limited to: an optional prehybridization step; a hybridization step; one or more wash steps to remove the labelled probe, an antibody, avidin or other ligand binding step, one or more wash steps to remove unbound ligand, additional steps, depending on the type of label used, to develop the label. Only the hybridization step and detection is actually required. An example of an in situ hybridization with fluorescent probes, FISH, can be found in Hughes S C and Krause H M. Biotechniques 24:530-532, (1998). An example of in situ hybridization using colorimetric in situ hybridization, CISH, is (Schroder, Hain and Sterflinger, Internatl. Microbiol. 3:183-186 (2000)).

An additional advantage to the HISH procedure is that the hybridization reaction can be monitored and recorded as it happens. Therefore, one can immediately monitor when the reaction has reached completion because the fluorescent signal (or any signal from a DM or a MM) will stop increasing with increased time of incubation. Therefore, there is no need to repeatedly run multiple step in situ reactions to determine the optimum time for hybridization. Such HISH methods are denoted as real time HISH.

In one embodiment, the analog probe complexes can be used to perform in situ experiments on any tissue type. For example, liver, kidney, muscle, and brain tissues may all be the subject of these in situ hybridization techniques. Nerve tissue may also be targeted, especially in combination the hybrid DMs described below. As will be appreciated by one of skill in the art, the location of examination is not a limitation on the present embodiments. Similarly, various components in the circulatory system may also be targeted for observation via these in situ techniques.

### EXAMPLE 9

This example demonstrates how HISH might be used. First, one will take a slide containing a fixed mounted tissue on it and add a suitable volume of 100 microliters of 1 nM-1 μM analog probe complex dissolved in 0.1 M NaCl, 0.01 M TRIS-HCL, 0.001 EDTA pH 7.5. The slide will be incubated at a suitable temperature, 37-50° C. Fluorescence will be monitored with the appropriate fluorescence imaging microscopy. Changes in fluorescence will indicate the binding of the probe 51 to the target sequence 65.

### EXAMPLE 10

This example demonstrates how HISH could be performed with live tissue. One will have the live tissue in a mounting condition. This condition will be one that would normally be used for physiological studies of the tissue or cells being studied. The analog probe complex, which will have been dissolved in the same physiological buffer or other appropriate solution, will then be added to the slide or viewing chamber to a final concentration of 1 nM-1 μM. The hybridization is followed by fluorescence imaging microscopy. Where ever there is an appropriate change in fluorescence in the sample, this will indicate the presence of the target sequence.

The analog probe complexes can also be used in a different type of analog probe complex-based hybridization assay. In this embodiment the analog probe complexes will have utility in improving assays used to detect, identify or quantitate the presence or amount of an organism or virus in a sample through the detection of target sequences associated with the organism or virus. (See: U.S. Pat. No. 5,641,631, entitled “Method for detecting, identifying and quantitating organisms and viruses” hereby incorporated by reference in its entirety). Similarly, this embodiment will also find utility in an assay used in the detection, identification or quantitation of one or more species of an organism in a sample (See U.S. Pat. No. 5,288,611, entitled “Method for detecting, identifying and quantitating organisms and viruses” hereby incorporated by reference in its entirety). This embodiment will also find utility in an assay used to determine the effect of antimicrobial agents on the growth of one or more microorganisms in a sample (See: U.S. Pat. No. 5,612,183, entitled “Method for determining the effect of antimicrobial agents on growth using ribosomal nucleic acid subunit subsequence specific probes” hereby incorporated by reference in its entirety). This embodiment will also find utility in an assay used to determine the presence or amount of a taxonomic group of organisms in a sample (See: U.S. Pat. No. 5,601,984, entitled “Method for detecting the presence of amount of a taxonomic group of organisms using specific r-RNA subsequences as probes” hereby incorporated by reference in its entirety).

When performing the method, it can be useful to use one or more unlabelled or independently detectable probes in the assay to thereby suppress the binding of the analog probe complex to a non-target sequence. The presence of the “blocking probe(s)” helps to increase the discrimination of the assay and thereby improve reliability and sensitivity (signal to noise ratio).

In certain embodiments, one target sequence 65 is immobilized to a surface by proper treatment of the sample. Immobilization of the nucleic acid can be easily accomplished by applying the sample to a membrane and then UV-crosslinking. For example, the samples can be arranged in an array so that the array can be sequentially interrogated with one or more analog probe complexes to thereby determine whether each sample contains one or more target sequence of interest. In one embodiment, the analog probe complex can be immobilized to a support and the samples sequentially interrogated to thereby determine whether each sample contains a target sequence of interest, as discussed in the arrays section above. The analog probe complexes can also be immobilized on an array which is then contacted with the sample of interest. Consequently, the sample can be simultaneously analyzed for the presence and quantity of numerous target sequences of interest wherein the composition of the analog probe complexes are judiciously chosen and arranged at predetermined locations on the surface so that the presence, absence or amount of particular target sequences can be unambiguously determined. Arrays of analog probe complexes are particularly useful because no second detection system is required since analog probe complexes can be self-indicating in some embodiments. Consequently, one embodiment is directed to an array comprising two or more support bound analog probe complexes suitable for detecting, identifying or quantitating a target sequence of interest.

MRI Based Analog Probe Complexes and Uses Thereof:

As discussed above, the actual signaling or detection method for determining whether or not the analog probe complex is still hybridized 10 or has located and bound to a target sequence 65 need not be fluorescence based.

The detectable marker and marker modifier can be based on magnetic resonance imaging (MRI) systems and detectable markers. MRI is based on the principles of nuclear magnetic resonance (NMR) and can be used for medical applications to produce high resolution images with anatomical detail and soft tissue contrast of the inside of animal bodies without damage to the organ being studied. Functional MRI (fMRI) is used to study the response of an organ to a particular stimulus. For example in the brain. fMRI is used to show which parts of the brain become active during a particular type of experience: stress, learning experience, sexual stimulation, etc. Advantages of MRI over parallel techniques like Positron Emission Tomography (PET) include the benefits that the resolution of MRI is much higher than PET, and that there is no need to inject a patient with short lived isotopes. However, one current disadvantage of MRI is that MRI gives pictures of general increases in physiological activity such as increased blood flow, and cannot target specific cellular functions like transcription, translation or enzyme activity.

To enhance the detection and specificity of specific tissue discrimination, lymphotrophic superparamagnetic nanoparticles were previously used as an imaging agent to visualize metastases in lymph nodes (N Engl J Med 348; 25). In that experiment, the differential accumulation of superparamagnetic particles in lymph nodes was used to distinguish malignant to normal lymph nodes. The diagnostic decision also depends on the shape and size of the tumor and therefore is only useful after the tumor has established itself for a significant period of time.

Using the methods described herein, a superparamagnetic detection marker is provided whose ability to be detected by MRI detection means is sensitive to the alteration of a molecular probe. In other words, rather than the fluorescent moieties described above being used as a detectable marker 50 and marker modifier 40, a MRI detectable marker 50 and a MRI marker modifier are used 40. This method can be aimed at detecting the presence of different signal molecules that indicate a cell's state of differentiation, gene expression, etc. which would be an early indicator of tissue pathology, cancer development, etc. This method can also be directed to detecting other markers of disease or infection and to detecting normal cellular processes which occur in vivo, for example in the brain or in the generalized nervous system. It can also be used for the detection of RNA, such as mRNA in tissues or cells.

A superparamagnetic detectable marker (SDM) 50 can be attached to a probe 20 and the signal of the SDM is shifted by a local magnetic field shielder 40, “B-field shielder,” in close proximity. This can be achieved through the use of the analog probe complex described above in FIG. 4A, with the appropriate DM and MM added. This can also be achieved with a PNA/D-DNA probe complex or a D-DNA/D-DNA probe complex. Any of the compositions and methods for keeping a DM and a MM together for a period of time and then separating in the presence of a target sequence could be used.

The reaction of the analog probe complex 10 with the target molecule 60 in the cell separates the superparamagnetic particle 50 from the B-field shielder 40 which, in some embodiments, results in a change in both the frequencies and phases of the superparamagnetic detectable marker. The superparamagnetic detectable marker can be a nanoparticle.

In one embodiment of the analog probe complex, the first segment 20 of the probe comprises PNA and is attached to a nanoparticle with nuclear spin, for example, a superparamagnetic ferromagnetism nanoparticle. The first segment further comprises a sequence 25 that is complementary to a specific target sequence 65 on one of the RNA members in the variety of RNA molecules being monitored. The second segment 30 of the analog probe complex 10, comprises a L-DNA sequence 35 that is complementary to the end of the first segment 28. The second segment 30 is attached to a marker modifier 40, for example an ordered magnetizer with an influence in the β-field; this marker modifier is referred to as a beta-field shielder. The beta-field shielder is appropriately placed on the second segment 30 to change the local magnetic field of the marker modifier 50 on the first segment 20, the PNA containing portion. For example, the β-field shielder will shift the MRI signals from the DM 50 to different radio frequencies and it also will significantly change the local dephasing environment to provide contrast media for MR imaging.

In order to facilitate the nucleation step that initiates the hybridization reaction between the target sequence and the probing segment 20, the first segment 20 can be longer than the second segment 30 on the end distal to the DM 50 β-shielder 40 pair. This helps to overcome hybridization problems caused by the different inherent chirality of single stranded RNA and a PNA/L-DNA helix. As described above, the RNA targets will hybridize with a PNA sequence that is complementary to their sequences. This will result in a displacement of the second segment 30, the L-DNA portion of the probe, because the PNA/RNA hybrid is more stable than the PNA/L-DNA hybrid and kinetically favored. The displacement of L-DNA from the hybrid probe separates the B-field shielder 40 from the nanoparticle moiety 50.

Examples of possible detectable moieties include nanometer size Fe₂O₃ or rare-earth heavy metals with superparamagnetic properties, such as Gadolinium (e.g., Gd-DTPA, diethylenetriaminepentaacetic acid). The same materials can be used for the beta-shielders. The DM can be an iron oxide based colloid, comprising nonstoichiometric microcrystalline magnetite cores that are coated with dextranes or siloxanes. Additionally, as will be appreciated by one of skill in the art, the MRI DM need not be limited to superparamagnetic particles, as traditional paramagnetic contrast agents can also be used.

### EXAMPLE 11

This example demonstrates one possible technique by which one can use the MRI detectable analog probe complexes. An analog probe complex 10 is prepared with a superparamagnetic detectable marker 50, a beta-shielder 40, and a probe sequence 25 that is specific to a target mRNA sequence 65 to be examined. The analog probe complex is administered to the patient and a sufficient amount of time is allowed to allow the analog probe complex to travel to the target of interest. Then a MRI is performed, allowing the actual detection of the target sequence, with minimal background signal to be observed.

Kits:

Another embodiment of the present invention is directed to kits suitable for performing an assay that detects the presence, absence or amount of one or more target sequence which can be present in a sample. The characteristics of analog probe complexes suitable for the detection, identification or quantitation of the amount of one or more target sequence have been previously described herein and are described further below. Furthermore, methods suitable for using the analog probe complex components of a kit to detect, identify or quantitate one or more target sequence which can be present in a sample are also described herein. In addition to the analog probe complexes of FIG. 4A, or similar such complexes which display a change in detectable signal upon binding of the probe to a target sequence, the kits described herein can also include the zip-coded analog complex probes, or the hybrid detectable markers described below. The kits can comprise one or more analog probe complexes and other reagents or compositions that are selected to perform an assay or otherwise simplify the performance of an assay. In one example, the kits contain sets of analog probe complexes, wherein each of at least two analog probe complexes of the set are used to distinctly detect and distinguish between the two or more different target sequences that can be present in the sample. Thus, the analog probe complexes of the set are preferably labelled with independently detectable markers so that each of the two or more different target sequences can be individually detected, identified or quantitated (for example, in a multiplex assay).

The kits can be directed to in vivo use or in vitro use, as well as other possible uses. Similarly, the kits can be directed to detection of any nucleotide sequence, genomic DNA or mRNA for example. Additionally, the kits can be for MRI use or for fluorescence based use: The kits can contain additional detectable markers, such as antibodies labelled with a fluorescent probe, so that detection can be mediated in that manner. The kits can also include instructions for a particular use or method of detecting the detectable marker or changes in the signal from the detectable marker. In another embodiment, the kits contain additional agents for the administration of the analog probe complexes. For example, cationic solutions as described herein can be included to allow increased uptake of the analog probe complexes into cells.

The kits can be specialized for a particular target cell type. For example, the kits can contain directed delivery vehicles that can transport the analog probe complexes to the desired cell type or organ of choice. Such compositions are known in the art and can include antibody associated compositions, where the antibodies are directed to a particular cell marker on the target cell. The kits can be directed to neuronal cells and can include appropriate delivery options for delivering the analog probe complexes to the neuronal cells. In one embodiment, the directed delivery compositions can be distinguish between certain targets as well. For example, in one embodiment, the directed delivery compositions can target axons over dendrites or vice versa. In another embodiment, the directed delivery compositions can target brain tissue over spinal cord tissue or vice versa. In one embodiment, the kits can contain multiple directed delivery compositions. In one embodiment, the kit is configured to allow various analog probe complexes to be associated with various directed delivery compositions, allowing the user to use the kit to administer multiple analog probe complexes to different targets if so desired. As will be appreciated by one of skill in the art, the kits can contain various compositions for the transfection of cells. In one embodiment, any of the ingredients for performing a transfection of cells can be included in the kits.

In the case of zip-coded analog probe complexes, the kit can comprise a detectable marker attached to a zip-coded L-DNA segment and sequence. The kit can further comprise another zip-coded L-DNA segment that can hybridize to the zip-coded L-DNA segment of the first segment. These two segments can be kept separate and the zip-coded section that is not associated with the DM kept in a condition to allow the ready addition of a PNA sequence, wherein said PNA sequence can be selected by the kit's user, and can be a probing sequence for determining the presence of a target sequence, for example. Thus, the kit can comprise a zip-coded L-DNA segment associated with a detectable marker and a complementary zip-coded L-DNA segment that is primed for the addition of a PNA segment, by a PEO/PEO polymer linker for example. The kit can include both the basic elements and the chemicals required to connect the elements to create a final zip-coded analog probe complex.

There can be several different samples of the same L-DNA segment that is attached to different DMs. For example, the kit can contain one container of zip-coded L-DNA segments attached to a red Q-dot and one container of zip-coded L-DNA segments attached to blue Q-dot. However, in this embodiment, all of the L-DNA sequences that are attached to the various DMs will have the same sequence. Similarly, there will be a container with a complementary zip-coded L-DNA sequence 105 that will hybridize to the DM labelled L-DNA sequence 115. Thus, while the connection of the zip-coded L-DNA sequence 105 to a PNA sequence 103 can be carried out in one reaction area, the final analog probe complex 190 can be labelled with any DM desired, for example, a blue Q-dot. This can be achieved by mixing the first segment 100, with the second segment 110 that has a particular DM 120, under conditions which allow hybridization. Similarly, a second PNA sequence 153 can be attached to a zip-L-DNA sequence 155, which can be the same sequence as the other L-DNA sequence 105, and labelled with a different colored DM 170 via a complementary L-DNA sequence 165. In another embodiment, a second PNA sequence 153 can be attached to a zip-L-DNA sequence 155 and labeled with a different colored DM 170 via a complementary L-DNA 16, whose zip-code identifies the color it is attached to. As will be apparent to one of skill in the art, one advantage of this embodiment is that the kit provides a means by which numerous PNA sequences can be hybridized to distinguishable DMs, thus allowing an inexpensive and rapid way to create probes with DMs. The user can supply the PNA sequence 103 or 153; a PNA sequence 103 or 153 can be supplied in the kit itself. Other possible embodiments involving zip-coded sequences are disclosed in U.S. Patent Application No. 60/584,799, filed Jun. 30, 2004, hereby incorporated by reference in its entirety.

Chimeric Analog Probe Complexes, or Zip-Coded Analog Probe Complexes:

The use of detectable markers, such as nanoparticles, (e.g., gold nano-particles and semi-conductor nanoparticles (quantum dots, Q-dots)) in cellular imaging and mRNA detection is complicated by the difficulty of attaching oligonucleotide probes to the nanoparticles. For example, commercially available Q-dots (for example from QuantumDot, Hayward, Calif.) labelled with streptavidin can only be used to do single color imaging. For multi-color imaging applications, one has to attach target specific probes to each of the detectable markers. This probe specific attachment can be an expensive manipulation since each probe type would require a connection step linking the probe to a particular detectable marker. Furthermore, cellular uptake of oligo labelled nanoparticles can be difficult as well. Thus, it may be desirable to have an alternative method or composition by which various DMs can be added to particular probe sequences.

An example of a composition that can allow the placement of a DM onto a probe sequence is shown in FIG. 5.

Two analog probe complexes 190 and 200 are shown in FIG. 5. These probes are referred to as chimeric configurational probe complexes, chimeric analog probe complexes, zip-coded analog probe complexes, or other similar terms. These probe complexes 190 and 200 allow for the attachment of various DMs 120 and 170 to a particular probe sequences 103 and 153.

The zip-coded analog probe complex 190 of FIG. 5 comprises a probe sequence 103 that can effectively hybridize to a target sequence 140. The target sequence 140 can be in various forms, for example, free in solution, attached to an array, or part of a larger sequence 130. The probe sequence 103 can comprise a PNA sequence. The probe sequence 103 is connected to a first zip-coded sequence 105. A zip-coded sequence can comprises a L-DNA sequence or other analog nucleic acid sequence that is not significantly susceptable to hybridization with D-DNA or normal RNA. By significant, it is meant that any amount of hybridization that does occur should not prohibit the hybridized pair to function as a probe. The probe sequence 103 and the first zip-coded sequence 105 are part of the first segment 100. The zip-coded analog probe complex 190 of FIG. 5 further comprises a second segment 110. The second segment comprises a second zip-coded sequence 115 that will effectively hybridize to the first zip-coded sequence 105. This second zip-coded sequence can comprise a L-DNA sequence. When both zip-coded sequences 115 and 105 comprise L-DNA sequences, the two sequences can form a duplex 112 that will be very stable under many different conditions and in the presence of most naturally occurring D-DNA and mRNA. The zip-coded duplex 112 comprises the first zip-coded sequence 105 and the second zip-coded sequence 115, which is also connected to a detectable marker 120.

In some embodiments, such as a kit, these elements are not yet connected. Thus, for example, a kit can comprise one or more first vials of DMs 120, a second vial of second zip-coded L-DNA sequences 115, and a third vial of first zip-coded L-DNA sequences 105. Thus, one can attach a PNA sequence 103 to the ingredients of the third vial to create the first segment 100, attach the desired DM 120 in the first vial to the second zip-coded L-DNA sequence 115 in the second vial, and then hybridize the second segment and the first segment together, to create the zip-coded L-DNA/PNA probe 190. Methods for these attachments are described herein. Instructions for said methods can be included in the kits. In one embodiment, the kit comprises a vial which contains DM 170 that are already associated with a L-DNA segment 165. The kit can contain multiple vials, each with a different DM or DMs associated with a different segment 165. In one embodiment, the segment 165 comprises a L-DNA nucleotide.

As discussed above, the detectable marker 120, can be any component which is observable, either directly, for example through fluorescence or MRI, or indirectly, for example through antibody binding and subsequent detection of the bound antibody.

The zip-coded sequences 115 and 105 or 165 and 155 can be made from PNA, D-DNA, or other types of sequences, as long as they are sufficiently “zip-coded.” Thus, the zip-coded analog probe complex need not include L-DNA or PNA and need not be chimeric. By “zip-coded” it is meant that the particular set of sequences creates a relatively stable duplex 112; thus, effectively connecting the first segment 100 to the second segment 110. This can be done in a variety of ways. For example, selecting the nucleotides to optimize the melting temperature of the duplex can achieve this. A sequence can be a zip-coded sequence if it will not substantially melt from its zip-coded complement at less than 50, 50-60, 60-70, 70-80, 80-90, 90-100, 100-120 degrees C. or more. A sequence can be zip-coded when the particular sequence selected will not substantially hybridize to a sequence that the zip-coded sequence will be exposed to. Thus, by knowing the possible sequences in a reaction mixture, for example in an organism, one is able to design a probe that will not hybridize to sequences in the reaction mixture or organism. In one example, the zip-coded sequence 105 or 155 are continuous PNA sequences with sequence 103 or 153 respectively. Thus, in one embodiment, the segment 100 only contains PNA sequences. As will be appreciated by one of skill in the art from the present disclosure, in some embodiments, the segment 115 can be either a complementary PNA sequence, a complementary L-DNA or L-RNA sequence. In one embodiment, the “zip-coded” sequence also allows the ready identification of a particular sequence to a particular detectable component. For example, the presence of a Q-dot attached to a known sequence will allow one to use the sequence and the Q-dot to index any object that binds to the Q-dot. In some of the present embodiments, the zip-coded sequence allows one to index a particular probe sequence with a particular DM.

A zip-coded sequence can be one that, when hybridized to its complement, will not substantially separate during use. For example, even without knowing the sequences in a given reaction, or the melting point of the candidate zip-coded probe, some analog sequences, such as L-DNA sequences, will only bind to other L-DNA sequences. Thus, any pair of sequences that are L-DNA sequences will serve as a zip-coded duplex, as long as substantially complementary L-DNA is not in the sample. In situations where sequences exist that can bind to L-DNA, then the above discussed aspects can be included to make the duplex a zip-coded duplex.

The length of the zip-coded L-DNA sequences 115 and 105, or the two sequences 115 and 105 that form the duplex 112, can vary depending upon the use. The L-DNA sequence can be from 1-30 nucleotides, 4-15, 6-12, or 8 nucleotides long, for example. Similarly, the length of the probe sequence will vary upon factors known to one of skill in the art. The length of the probe sequence 103 can be from 2-30, 4-15, 6-10 or 8 nucleotides long, for example. The sequence 105 and the probe sequence 103 can be connected via a chemical moiety that allows flexibility. There can be a linker inserted between the sequence 105 and the probe sequence 103. The two sequences 105 and 103 can be connected by any means which will allow the probes to function. The sequence 105 and the sequence 103 can be connected via a PEO/PEO connection, for example. Each of the sequences or segments can comprise additional natural or analog nucleotides; thus, for example, not all of the nucleotides between sequence 105 and sequence 115 need to hybridize together. The only requirement is that the duplex is sturdy enough for the probe to function for its intended purpose. The DM 120 can be positioned at the end of the sequence 115 away from the probe sequence 103; alternatively, the DM 120 can be attached to the sequence 115 at the end closest to the probe sequence 103.

The sequence 115 can comprise analog nucleotides that fluoresce with the need for an independent DM 120; thus, the DM will be inherent in the sequence 115. As discussed herein, the DM 120 can comprise any detectable molecule. Unlike some embodiments of the self-indicating analog probe complexes, the DM for these embodiments need not be altered by a MM, and thus, can include other DMs as well. Other examples of DMs include quantum dots (Q-dots) or any fluorophore in general. There can be situations involving multiple DMs in a single solution, in such cases, FRET can be employed, assuming the distances between the detectable markers is appropriate for revealing the desired information. Thus, FRET appropriate DMs can be employed.

The sequence 155 can be attached to the opposite end of the probe sequence 153 allowing the fourth segment 160 and the second DM 170 to be positioned closely to the second segment 110 and the first DM 120. This allows for FRET based monitoring methods to be used to follow co-localization of the two zip-coded analog probe complexes. Thus, for example, the two DM can be similar to the DM and MM pairings described herein. As will be appreciated by one of skill in the art, the proximity of the first duplex 112 with the second duplex 162, can allow for some of the segments to exchange between the complexes; however, this is not important, as long as both DMs are associated with the same segment 130 in some manner.

The zip-coded analog probe complex can have a complementary zip-coded sequence 115 of L-DNA that binds to a zip-coded L-DNA sequence 105 that is part of a probe PNA sequence 103. The probe PNA portion 103 will hybridize to a target sequence 140. The zip-coded L-DNA sequence 115 links the detectable marker 120 to the PNA probe sequence 103 by forming a duplex structure 112 with the complementary zip-code L-DNA sequence 105. The zip-coded analog probe complex 190 is useful in complex environments, for example, for in situ hybridization experiments. Thus, some embodiments include in situ hybridization methods using the zip-coded analog probe complex. This generally allows for any L-DNA sequence to serve as a zip-coded sequence, as L-DNA sequences are not present in organisms normally. Additionally, this allows for a two color detection scheme, e.g. two separate zip-coded analog probe complexes, to be created, as the same L-DNA sequences can be used on different PNA sequences 103 and 153 and with different DMs 120 and 170, reducing the amount of work to associate different DMs with a single probe sequence, or with various probe sequences that are connected to a single L-DNA sequence 105. In one embodiment, the zip-coded segment attached to each DM is unique, allowing a high degree of customization of association of the various DMs 170 and 120 to various probe sequences 153 and 103. The sequence 165 can be different from the sequence 115, and the sequence 155 can be different from the sequence 105. In such an embodiment, each DM 170 and 120 may be associated with an effectively unique sequence 165 and 115 and each complementary L-DNA sequence 155 and 105 will also be different from each other. In such an embodiment, the uniqueness of the sequences 165 and 115 will allow for the identification of multiple target sequences, simply by adding a relatively unique sequence 165 to the particular DM 170. Of course, as described above, the uniqueness of the probe sequence itself can also function to allow the association of a DM 170 with a target sequence 180 to indicate the presence of a unique target sequence.

The targeted sequence can be any nucleotide or set of nucleic acids. For example, DNA, rRNA, or mRNA can be targeted. The target sequence can be from any source, e.g., genomic or cDNA, and can include analog or artificial nucleic acids as well.

A co-localization detection scheme can involve two zip-coded analog probe complexes 190 and 200 that will co-hybridize to two desired target sequences 140 and 180. The first zip-coded analog probe complex 190 is as described above. The second zip-coded analog probe complex 200 can be similar, in that it has a third sequence 155 and a fourth sequence 165 that form a duplex 162. The sequence 165 can be the same as the sequence 115, and the sequence 155 can be the same as the sequence 105. Additionally, the sequence 155 is connected to a probe sequence 153 to form a third segment 150. The probe sequence 153 will hybridize to a different target sequence 180 than the first probe complex 190. Additionally, the sequence 165 is part of a fourth segment 160 that is connected to a second DM 170.

These two target sequences 180 and 140 can be located next to each other on a single nucleotide 130, such as mRNA. Thus, the movement of these co-localized target sequences 180 and 140 can be observed. One of probes 190 is labelled with a blue color Q-dot and another 200 is labelled with red color Q-dot 170, for example. In general Q-dots will randomly distribute in a cell, moving around independently of each other. However, upon hybridization both probes will become attached to the same nucleotide strand 130. As a result, the red 170 and blue 120 Q-dots become closely associated with each other and move together in the cell. By recording the trajectories of co-localized Q-dots in the cell, both mRNA/PNA hybridization and mRNA movement can be followed in the cell. Of course, as will be appreciated by one of skill in the art, the target sequences 180 and 140, need not be mRNA.

This co-localization technique allows for one to largely ignore the background signal from the DMs, as only those areas showing signals from both DMs 170 and 120, will be the desired set of target sequences. Thus, a set of zip-coded analog probe complexes can be used for any of the methods or in any of examples described above as only co-localized probe will be monitored. Thus, for example, the set of these probes can be used for HISH described above. This is simplified more in embodiments in which FRET occurs between each of the zip-coded analog probe complexes, as only those probes that fluoresce via the excitation of the donor will be the target sequence.

The target sequences 103 and 153 can be made of PNA and the zip-code sequences 105, 115, 155, and 165 are L-DNA, to create probes that are resistant to cellular degradation through the action of cellular nucleases. The zip-code sequences attached to Q-dots 115 and 165 are L-DNA sequences complementary to the zip-code L-DNA sequences 105 and 155 that are attached to the probe sequences 103 and 153.

The zip-coded analog probe complexes (APCs) can be used to determine the amount of target sequence present in a sample.

Typically, cellular uptake of PNA is inefficient because the back bone has a neutral charge. The chimeric PNA and L-DNA zip-coded probe should increase cellular update since L-DNA has negative charges, as does normal D-DNA. In addition, the stereochemistry of L-DNA insures that it will not hybridize to mRNA and gDNA in cells; such a reaction would interfere with the detection procedure.

Some advantages of the embodiments described above include (1) only two generic zip-coded L-DNA segments attached to Q-dots need to be manufactured since they can be associated to various probe sequences via hybridization; (2) the cellular uptake efficiency will increase for chimeric PNA/L-DNA probes; and (3) L-DNA sequences will not interact with mRNA and gDNA in cells. However, as will be appreciated by one of skill in the art, not every embodiment will have all or any of these advantages, and they can have other advantages as well.

As will be understood by one of skill in the art, the number of zip-coded analog probe complexes is not limited to 2 at a time. For example, in one embodiment, there can be 3, 4, 5-10, 10-20, 20-40, 40-100, thousands or more of the zip-coded analog probe complexes. Likewise, as will be appreciated by one of skill in the art, the number of DMs on a single zip-coded analog probe complex is not limited to only one. For example, there may be 2, 3, 4, 5, 6-10, or any number of DMs, as long as the segments and sequences can function as described. One possible reason to have multiple DMs is that it will allow a greater degree of customization of the signature of the entire analog probe complex. In one embodiment, DM or MM are also included on the PNA segment to allow for additional distinctions between segments to be made. For example, a DM 170 can be placed on the probe sequence 153, to allow for interactions such as FRET to occur, for greater customization of the fluorescent signature of each analog probe complex. As appreciated by one of skill in the art, much of the description of the self indicating analog probe complexes can apply equally well to various embodiments of the zip-coded analog probe complexes. Other possible embodiments involving zip-coded sequences are disclosed in U.S. Patent Application No. 60/584,799, filed Jun. 30, 2004, hereby incorporated by reference in its entirety.

### EXAMPLE 12

This example demonstrates one method by which several of the zip-coded analog probe complexes could be used on a single cell. First, second, and third zip-coded analog probe complexes are administered to a group of cells via a standard transfection method. The first zip-coded analog probe complex comprises a green Quantum-dot and a probe sequence that hybridizes to a mRNA target sequence X. The second zip-coded analog probe complex comprises a red Quantum-dot and a probe sequence that hybridizes to a mRNA target sequence Y. The third zip-coded analog probe complex comprises a blue Quantum-dot and a probe sequence that hybridizes to a mRNA target sequence Z. All three target sequences are on the same target segment of mRNA. After the zip-coded analog probe complexes are allowed to bind to their target sequences, the cells are observed under a microscope to track the movement of the three target sequences. The excess zip-coded analog probe complexes need not be washed away, since one can separate the unbound probe from the bound probe by following fluorescent signals that show all three fluorescent characteristics from the green, red, and blue quantum dots. The movement of the target segment of mRNA can then be observed throughout the cell. Additionally, the conditions of the cells may then be altered and the movement and presence of the mRNA target segment can then be monitored by tracking the labelled mRNAs that have the red, blue and green Quantum-dots attached to them.

Hybrid Detectable Marker:

Nanoparticles, such as gold nanoparticles and semiconductor nanoparticles (for example, quantum dots or Q-dots) have been used for MRI organ imaging, tumor detection, cellular imaging and macromolecular detection. Superparamagnetic nanoparticles are useful in MRI platforms; Q-dots are used with microscopes or fluorescence detectors. In some of the embodiments disclosed herein, fluorescent Q-dots have the added properties that they are stable against photobleaching; they can be excited by a single wavelength that is independent of the emission wavelength; they can be tuned for precise wavelength of fluorescence; and they have narrow, symmetric emission peaks. Each of these methods of detection, MRI and fluorescence, has applications that may overlap in the applied arena. MRI is a non-invasive method of imaging internal structures in the body. However, MRI is not suitable for the high resolution of histological examination. At the cellular level fluorescent microscopy combined with specific fluorescing probes can be used to localize probe detection to the subcellular level and even submolecular level. In some situations, it is advantageous to combine high resolution whole organism imaging with cellular level probes. For example, in the clinical area one may wish to examine the localization of a probe to a particular diseased area in the whole body of an animal and then subsequently examine histological preparations from a biopsy of a specific organ. By employing a hybrid DM that has both fluorescent and superparamagnetic properties one will be able to detect the localization of a hybrid nanoparticle in a specific organ using MRI and then subsequently use fluorescence microscopy to study the distribution of the hybrid nanoparticles in a histological amount of a biopsy from the targeted tissue.

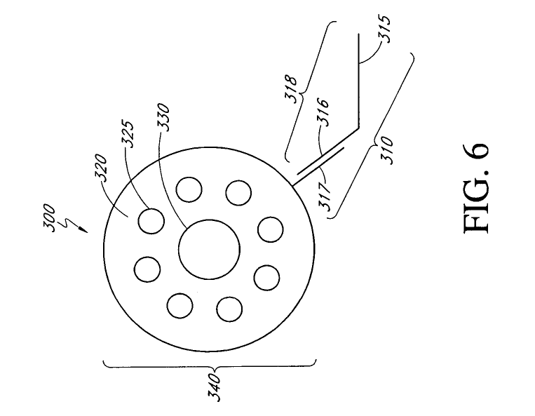

One embodiment of a hybrid long-range/short-range detectable marker is shown in FIG. 6. By long-range, it is meant that the detectable marker is detectable over distances in the range of 1-10, 10-100, 100-200, 200-500, 500-800, 80-1000, 1000-2000, millimeters or more. Short range means 100-1, 1-0.1, 0.1-0.01, 0.01-0.001, 0.001 to 10⁻⁵, 10⁻⁵ to 10⁻⁶, 10⁻⁶ to 10⁻⁷ millimeters or less. Both of these terms are defined by the standard means by which DMs are usually observed. Thus, DMs detected by traditional MRI probes will be long range DM, and DMs detected by traditional fluorescence techniques will be short range DMs. For convenience, superparamagnetic DMs are discussed herein, with the understanding that any MRI detectable agent, such as paramagnetic agents, could also be used. Additionally, the long range detection of DM is not always performed via a MRI, for example, the long range DM could be an isotope and detection could be achieved through the detection of the isotope.

The hybrid detectable marker 340 can comprise one detectable marker 330 that is readily detectable over long-ranges, and one detectable marker that is detectable over short-ranges 325 at high resolution. The hybrid detectable marker 340 can comprise a Q-dot 325 and superparamagnetic nanoparticle 330, for example. This hybrid can be used in both MRI and fluorescence studies, thus allowing both the broad detection of a probe throughout an entire system and the detection of a probe in a localized area, within a cell or a part of a cell for example.

The hybrid DMs 340 can be attached to specific probes (nucleic acids, antibodies, etc.) to target specific molecules on cell surfaces or inside cells. These probes can be zip-coded PNA/L-DNA probes 310. These zip-coded PNA/L-DNA probes comprise a zip-coded L-DNA section which comprises two segments of zip-coded L-DNA that will hybridize to each other 316 and 317. Additionally, one of the segments 317 of the zip-coded L-DNA is connected to the hybrid DM 340. The other segment or sequence 316 is part of a larger segment 318 that contains a probe sequence 315 that can localize the zip-coded L-DNAs and everything to which they are bound to the target sequence. The hybrid DMs can also be attached to any type of localizing particle. For example, the hybrid DMs can be attached to antibodies or other compounds that localize to a desired location.

The fluorescent moieties 325 and the superparamagnetic particle can be kept together through the use of a shell 320. Additionally, the L-DNA zip-coded sequence 317 can also be attached to the shell 320. The shell 320 can be made of any material that allows a sufficient amount of fluorescent signal and MRI detectable signal through the hybrid DM 340. The shell can be a polymer, for example, polystyrene. The superparamagnetic core can be from 10-50 nm in diameter. The superparamagnetic core can be made from superparamagnetic iron oxide. In one embodiment, the Q-dots 325 in the shell layer 320 are from 2-8 nm in diameter. In one embodiment, the polymer coating 320 around the core 330 is from 50-300 nm in diameter.

The structure can be such that the superparamagnetic component forms a core 330 of the DM 340 which is surrounded by a polymer shell 320 and fluorescent moieties 325. More than one type of fluorescent moiety can be used, as it will form a more unique signal to be observed from the hybrid DM. In one embodiment, FRET occurs between the more than one fluorescent moiety in the hybrid DM, allowing excitation at one wavelength and observation at a very different wavelength, allowing one to avoid certain types of background noise.

The hybrid DM 340 can be combined with a zip-coded L-DNA analog probe complex 310 to result in an very stable connection between the hybrid DM 340 and a probe 310, but a connection that can be manipulated for a particular combination of DM and probe sequence 315, which will depend upon the desired target sequence. A particular probe sequence 315 can be added to a particular hybrid DM via this zip-coded pairing.

The hybrid DM 340 and the zip-coded L-DNA attached hybrid DM 300, can be used in any of the methods, kits, or other compositions described herein, as will be appreciated by one of skill in the art. One useful method is described in the example below.

### EXAMPLE 13

This example provides a use of hybrid DM. The hybrid DM will be attached to a specific antibody that binds a cellular antigen at cell surface. This hybrid DM will be administered to a patient. Following a period of time sufficient to allow for the localization of the hybrid DM to a target site, a MRI will be used to localize the binding areas of the hybrid DMs within the body. This will be followed by a biopsy, which will be followed by a histological examination with fluorescence microscopy to identify the cell type or area within an organ where the DMs are localized.

Self Indicating Zip-Coded Analog Probe Complexes (SIZAPC):

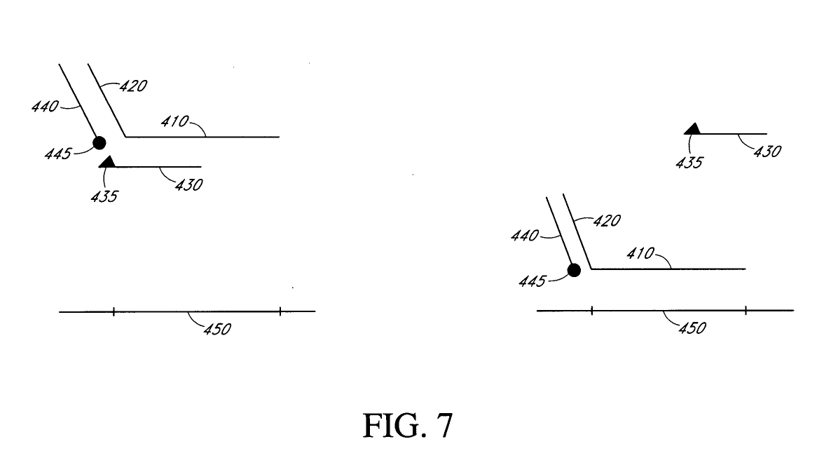

As will be appreciated by one of skill in the art, there are many combinations of analog probe complexes (APC) that are possible from the teachings herein. For example, the self-indicating probe can also comprise a zip-coded section. An example of this is shown in FIG. 7. In one embodiment, the SIZAPC is similar to the APC shown in FIG. 4A. As such, only a general overview is shown here, although all of the complexities of the embodiments described above can apply equally well here. The SIZAPC comprises a first sequence 410 that comprises a probe sequence and can comprise PNAs. The first sequence 410 is hybridized to a complementary sequence 430, that has a MM 435 attached to it, for example, a fluorescent quencher. The first sequence 410 is attached to another sequence 420. This sequence 420 is part of a zip-coded duplex, and can comprise L-DNA. The sequence 420, is hybridized, stably, to the other part of the zip-coded L-DNA duplex 440. The sequence 440 is attached to a DM 445, for example a fluorescent donor. As shown on the left side of FIG. 7, when sequence 430 and sequence 410 are hybridized, the DM 445 and the MM 435 are in close proximity to each other. However, when the sequence 410 binds to a target sequence 450, forcing the sequence 430 to leave, the distance between the DM 445 and the MM 435 increases. As described above, this will result in a change in signal which can be monitored for as an indicator of the presence of the target sequence.

PNA Synthesis:

Methods for the chemical assembly of PNAs are well known (See: U.S. Pat. No. 5,539,082, 5,527,675, 5,623,049, 5,714,331, 5,736,336, 5,773,571 or 5,786,571 (all of which are hereby incorporated, in their entireties, by reference). Chemicals and instrumentation for the support bound automated chemical assembly of Peptide Nucleic Acids are now commercially available. Chemical assembly of a PNA is analogous to solid phase peptide synthesis, wherein at each cycle of assembly the oligomer possesses a reactive alkyl amino terminus which is condensed with the next synthon to be added to the growing polymer. Because standard peptide chemistry is utilized, natural and non-natural amino acids are routinely incorporated into a PNA oligomer. Because a PNA is a polyamide, it has a C-terminus (carboxyl terminus) and an N-terminus (amino terminus). For the purposes of the design of a hybridization probe suitable for antiparallel binding to the target sequence (the preferred orientation), the N-terminus of the probing nucleobase sequence of the PNA probe is the equivalent of the 5′-hydroxyl terminus of an equivalent DNA or RNA oligonucleotide.

L-DNA Synthesis:

L-form and D-form phosphoramidite nucleosides can be prepared and used in oligonucleotide synthesis according to known procedures and methods of sugar and nucleobase protection and phosphitylation of the respective nucleosides. D-form nucleosides are derived from naturally occurring D-DNA sources. L-form phosphoramidite nucleosides can be prepared by any suitable synthetic method. For example, L-form phosphoramidite nucleosides can be prepared from L-ribose, which can be derived from L-xylose in a series of steps (Chu, U.S. Pat. No. 5,753,789; Fujimori Nucleosides & Nucleotides 11:341-49 (1992); Beigelman, U.S. Pat. No. 6,251,666; Furste, WO 98/08856).

L-DNA and PNA, when needed to, can be covalently connected in any number or ways. For example, via a polymer linker, such as PEO to PEO. In another embodiment, the L-DNA and PNA linked molecule, can be produced directly on a synthesizer.

Probe Sequence:

The probing nucleobase sequence, or “probe sequence,” of a probe is the sequence recognition portion of the construct. Therefore, the probe sequence is designed to hybridize to at least a portion of the target sequence. The sequence can hybridize to the entire target sequence. The probe sequence can be a non-polynucleotide. The probe sequence can be composed exclusively of PNA units. The length of the probe sequence (and therefore minimum length of the segment) can be chosen such that a stable complex is formed between the analog probe complex and the target sequence sought to be detected, under suitable hybridization conditions. The probe sequence can be any length, and will depend upon the particular application, as will be appreciated by one of skill in the art. The probe sequence of a PNA oligomer can have a length of between 1 and 40 PNA subunits, including those described above and including 8 to 18, or 8-12 subunits in length. The length of the entire probe segment can also be exactly the same as the length of the target sequence. The length of the probe segment can also be longer than the length of the L-DNA sequence by any amount. For example, the PNA segment, and probing sequence, can be 1, 2, 3, 4, 5, 6, 7, 8, 9, 10, 11-20, 20-30 or greater units longer than the number of nucleic acids present in L-DNA sequence.

The probe sequence can generally have a nucleobase sequence which is complementary to the target sequence. Alternatively, a substantially complementary probing sequence might be used since it has been demonstrated that greater sequence discrimination can be obtained when utilizing probes wherein there exists a single point mutation (base mismatch) between the probing nucleobase sequence and the target sequence (See: Guo et al., Nature Biotechnology 15: 331-335 (1997), Guo et al., WO97/46711; and Guo et al., U.S. Pat. No. 5,780,233, hereby incorporated in their entireties by reference).

Labels and FRET:

The labels attached to the analog probe complexes can comprise a set (hereinafter “Probe Set(s)”) of energy transfer moieties comprising at least one energy donor and at least one energy acceptor moiety. The Probe Set can include a single donor moiety and a single acceptor moiety. A Probe Set can also contain more than one donor moiety and/or more than one acceptor moiety. The donor and acceptor moieties operate such that one or more acceptor moieties accepts energy transferred from the one or more donor moieties or otherwise quench signal from the donor moiety or moieties. Embodiments of the energy transfer moieties operate by both FRET and non-FRET mechanisms.

General labeling can be accomplished using any one of a large number of known techniques employing known labels, linkages, linking groups, reagents, reaction conditions, and analysis and purification methods. Labels include light-emitting or light-absorbing compounds which generate or quench a detectable fluorescent, chemiluminescent, or bioluminescent signal (Kricka, L. in Nonisotopic DNA Probe Techniques, Academic Press, San Diego, pp. 3-28 (1992)). Fluorescent reporter dyes useful for labeling biomolecules include fluoresceins (for example, U.S. Pat. Nos. 5,188,934; 5,654,442; 6,008,379; 6,020,481), rhodamines (for example, U.S. Pat. Nos. 5,366,860; 5,847,162; 5,936,087; 6,051,719; 6,191,278), benzophenoxazines (for example, U.S. Pat. No. 6,140,500), energy-transfer dye pairs of donors and acceptors (for example, U.S. Pat. Nos. 5,863,727; 5,800,996; 5,945,526), and cyanines (for example, Kubista, WO 97/45539), as well as any other fluorescent label capable of generating a detectable signal. Specific examples of fluorescein dyes include 6-carboxyfluorescein; 2′,4′,1,4,-tetrachlorofluorescein; and 2′,4′,5′,7′,1,4-hexachlorofluoresc-ein (e.g., U.S. Pat. No. 5,654,442). Another class of labels are hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, “Chemical methods for 5′ non-isotopic labelling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

In one embodiment, the donor moiety is a fluorophore. Examples of fluorophores are derivatives of fluorescein, derivatives of bodipy, 5-(2′-aminoethyl)-aminonaphthalene-1-sulfonic acid (EDANS), derivatives of rhodamine, Cy2, Cy3, Cy 3.5, Cy5, Cy5.5, texas red and its derivatives. In principle any fluorophore can be used. Any fluorophore described in the Ninth Edition of the Handbook of Fluorescent Probes and Research Products, (Edited by Richard P. Haugland, (2002) hereby incorporated in its entirety by reference) can be used, with particular emphasis on the fluorescent molecules in chapter 1. Though the previously listed fluorophores might also operate as acceptors, the acceptor moiety can be a quencher moiety. The quencher moiety can be a non-fluorescent aromatic or heteroaromatic moiety. For example, the quencher moiety can be 4-((-4-(dimethylamino)phenyl)azo) benzoic acid (dabcyl).

Examples of possible methods for attaching a fluorescent probe to a nucleic acid are also provided in the Handbook of Fluorescent Probes and Research Products, Ninth Edition, with special emphasis given to chapter 8, sections 8.1, “nucleic acid stains,” and section 8.2, “labeling oligonucleotides and nucleic acids.” Additionally, labels can be attached though sulfur groups to maleimide groups. Alternatively, labels are attached through additional linkers, such as streptavidin to biotin, which is connected to the nucleic acid sequence, or via a dye-labelled antibody. Thus, the attachment of the label to the segment can be either covalent or noncovalent.

Chemical labeling of a PNA segment or sequence can be analogous to peptide labeling. Because the synthetic chemistry of assembly is essentially the same, any method commonly used to label a peptide can be used to label a PNA segment or sequence. For example, the N-terminus of the polymer is labelled by reaction with a moiety having a carboxylic acid group or activated carboxylic acid group. One or more spacer moieties can optionally be introduced between the labeling moiety and the probing nucleobase sequence of the oligomer. Generally, the spacer moiety is incorporated prior to performing the labeling reaction. However, the spacer can be embedded within the label and thereby be incorporated during the labeling reaction.

The C-terminal end of the probing nucleobase sequence can be labelled by first condensing a labelled moiety with the support upon which the PNA is to be assembled. Next, the first synthon of the probing nucleobase sequence can be condensed with the labelled moiety. Alternatively, one or more spacer moieties can be introduced between the labelled moiety and the oligomer (e.g. 8-amino-3,6-dioxaoctanoic acid). Once the analog probe complex is completely assembled and labelled, it is cleaved from the support deprotected and purified using standard methodologies.

The labelled moiety can be a lysine derivative wherein the epsilon-amino group is modified with a donor or acceptor moiety. For example, the label could be a fluorophore such as 5(6)-carboxyfluorescein or a quencher moiety such as 4-((4-(dimethylamino)phenyl)azo)benzoic acid (dabcyl). Condensation of the lysine derivative with the synthesis support would be accomplished using standard condensation (peptide) chemistry. The alpha-amino group of the lysine derivative would then be deprotected and the probing nucleobase sequence assembly initiated by condensation of the first PNA synthon with the alpha-amino group of the lysine amino acid. As discussed above, a spacer moiety could optionally be inserted between the lysine amino acid and the first PNA synthon by condensing a suitable spacer (e.g., Fmoc-8-amino-3,6-dioxaoctanoic acid) with the lysine amino acid prior to condensation of the first PNA synthon of the probing nucleobase sequence.

Alternatively, a functional group on the assembled, or partially assembled, polymer can be labelled with a donor or acceptor moiety while it is support bound. This method requires that an appropriate protecting group be incorporated into the oligomer to thereby yield a reactive functional to which the donor or acceptor moiety is linked, but has the advantage that the label (e.g., dabcyl or a fluorophore) can be attached to any position within the polymer including within the probing nucleobase sequence. For example, the epsilon-amino group of a lysine could be protected with a 4-methyl-triphenylmethyl (Mtt), a 4-methoxy-triphenylmethyl (MMT) or a 4,4′-dimethoxytriphenylmethyl (DMT) protecting group. The Mtt, MMT or DMT groups can be removed from PNA (assembled using commercially available Fmoc PNA monomers and polystyrene support having a PAL linker; PerSeptive Biosystems, Inc., Framingham, Mass.) by treatment of the resin under mildly acidic conditions. Consequently, the donor or acceptor moiety can then be condensed with the epsilon.-amino group of the lysine amino acid. After complete assembly and labeling, the polymer is then cleaved from the support, deprotected and purified using well known methodologies.

The donor or acceptor moiety can be attached to the polymer after it is fully assembled and cleaved from a support. This method is useful where the label is incompatible with the cleavage, deprotection or purification regimes commonly used to manufacture the oligomer. By this method, the PNA will generally be labelled in solution by the reaction of a functional group on the polymer and a functional group on the label. Those of ordinary skill in the art will recognize that the composition of the coupling solution will depend on the nature of oligomer and the donor or acceptor moiety. The solution can comprise organic solvent, water or any combination thereof. Generally, the organic solvent will be a polar non-nucleophilic solvent. Non-limiting examples of suitable organic solvents include acetonitrile, tetrahydrofuran, dioxane, methyl sulfoxide and N,N′-dimethylformamide.

Generally the functional group on the polymer to be labelled will be an amine and the functional group on the label will be a carboxylic acid or activated carboxylic acid. Non-limiting examples of activated carboxylic acid functional groups include N-hydroxysuccinimidyl esters. In aqueous solutions, the carboxylic acid group of either of the PNA or label (depending on the nature of the components chosen) can be activated with a water soluble carbodiimide. The reagent, 1-(3-dimethylaminopropyl)-3-ethylcarbodiimide hydrochloride (EDC), is a commercially available reagent sold specifically for aqueous amide forming condensation reactions.

Labelled chimeric configurational oligonucleotides can be formed by coupling a reactive linking group on a label, e.g., a quencher moiety, with the chimeric configurational oligonucleotide in a suitable solvent in which both are soluble or appreciably soluble, using methods well-known in the art. For labelling methodology, see Hermanson, Bioconjugate Techniques, ((1996) Academic Press, San Diego, Calif. pp. 40-55, 643-71; Garman, 1997, Non-Radioactive Labelling: A Practical Approach, Academic Press, London. Crude), labelled chimeric configurational oligonucleotides can be purified away from any starting materials or unwanted by-products, and stored dry or in solution for later use, preferably at low temperature.

The label can bear a reactive linking group at one of the substituent positions, e.g., an aryl-carboxyl group of a quencher, or the 5- or 6-carboxyl of fluorescein or rhodamine, for covalent attachment through a linkage. The linkage that links a label to a chimeric configurational oligonucleotide preferably should not (i) interfere with hybridization affinity or specificity, (ii) diminish quenching, (iii) interfere with primer extension, (iv) inhibit polymerase activity, or (v) adversely affect the fluorescence, quenching, capture, or hybridization-stabilizing properties of the label. Electrophilic reactive linking groups form a covalent bond with nucleophilic groups such as amines and thiols on a polynucleotide. Examples of electrophilic reactive linking groups include active esters, isothiocyanate, sulfonyl chloride, sulfonate ester, silyl halide, 2,6-dichlorotriazinyl, phosphoramidite, maleimide, haloacetyl, epoxide, alkylhalide, allyl halide, aldehyde, ketone, acylazide, anhydride, and iodoacetamide. Active esters include succinimidyl (NHS), hydroxybenzotriazolyl (HOBt) and pentafluorophenyl esters.

An NHS ester of a label reagent can be preformed, isolated, purified, and/or characterized, or it can be formed in situ and reacted with a nucleophilic group of a chimeric configurational oligonucleotide. A label carboxyl group can be activated by reacting with a combination of: (1) a carbodiimide reagent, e.g. dicyclohexylcarbodiimide-, diisopropylcarbodiimide, EDC (1-ethyl-3-(3-dimethylaminopropyl)carbodiim-ide); or a uronium reagent, e.g. TSTU (O-(N-Succinimidyl)-N,N,N′,N′-tetra-methyluronium tetrafluoroborate, HBTU (O-benzotriazol-1-yl)-N,N,N′,N′-tetr-amethyluronium hexafluorophosphate), or HATU (O-(7-azabenzotriazol-1-yl)-N-,N,N′,N′-tetramethyluronium hexafluorophosphate); and (2) an activator, such as HOBt (1-hydroxybenzotriazole) or HOAt (1-hydroxy-7-azabenzotriazo-le; and (3) N-hydroxysuccinimide to give the NHS ester.

One example of a non-nucleosidic phosphoramidite label reagent has the general formula VII, found in U.S. Patent Publication 2003/0198980, published Oct. 23, 2003 to Greenfield et al., page 14, paragraph 141. An alternative phosphoramidite labelling reagent is structure VIII, paragraph 143, of the same reference.

A phosphoramidite label reagent VII or VIII reacts with a hydroxyl group, e.g. 5′ terminal OH of a chimeric configurational oligonucleotide covalently attached to a solid support, under mild acid activation, e.g. tetrazole, to form an internucleotide phosphite group which is then oxidized to an internucleotide phosphate group. In some instances, the phosphoramidite label reagent contains functional groups that require protection either during the synthesis of the reagent or during its subsequent use to label a chimeric configurational oligonucleotide. The protecting group(s) used will depend upon the nature of the functional groups, and will be apparent to those having skill in the art (Greene, T. and Wuts, P. Protective Groups in Organic Synthesis, 2nd Ed., John Wiley & Sons, New York, 1991). The label will be attached at the 5′ terminus of the oligonucleotide, as a consequence of the common 3′ to 5′ direction of synthesis method with 5′-protected, 3′-phosphoramidite nucleosides. Alternatively, the 3′ terminus of an oligonucleotide can be labelled with a phosphoramidite label reagent when synthesis is conducted in the 5′ to 3′ direction with 3′-protected, 5′ phosphoramidite nucleosides, (Vinayak, U.S. Pat. No. 6,255,476).

Other phosphoramidite label reagents, both nucleosidic and non-nucleosidic, allow for labelling at other sites of a chimeric configurational oligonucleotide, e.g. 3′ terminus, nucleobase, internucleotide linkage, sugar. Labelling at the nucleobase, internucleotide linkage, and sugar sites allows for internal and multiple labelling.

Aswill be appreciated by one of skill in the art, donor or acceptor, marker or marker modifier moieties can be positioned on either the PNA segment or the L-DNA segment.

Fluorescent Interactions:

Transfer of energy can occur through collision of the closely associated moieties of a Probe Set or through a nonradiative process such as fluorescence resonance energy transfer (FRET). For FRET to occur, transfer of energy between donor and acceptor moieties of a Probe Set requires that the moieties be close in space and that the emission spectrum of a donor(s) have substantial overlap with the absorption spectrum of the acceptor(s) (See: Yaron et al. Analytical Biochemistry, 95: 228-235 (1979) and particularly page 232, col. 1 through page 234, col. 1; additionally see pages 25 and 26 of the Ninth Edition of the Handbook of Fluorescent Probes and Research Products, which generally discloses FRET requirements, how to determine the Forster radius (R₀) and typical Forster radii for common FRET pairs, such as Fluorescein and tetramethylrhodamine, IAEDANS and Fluorescein, EDANS and Dabcyl, Fluorescein and Fluorescein, BODIPY FL and BODIPY FL, Fluorescein and QSY7 or QSY 9 dyes). It is also possible to use nanoparticles, such as Q-dot (quantum dots) as fluorophores to further increase the low detection limit (LOD). The universal dark quenchers, such as silver/golden nanoparticles, can also be used yielding even better quench efficiency.

Non-FRET interactions can also occur. In one embodiment, this is collision mediated (radiationless) energy transfer. This can occur between very closely associated donor and acceptor moieties whether or not the emission spectrum of a donor moiety(ies) has a substantial overlap with the absorption spectrum of the acceptor moiety(ies) (See: Yaron et al., Analytical Biochemistry, 95: 228-235 (1979) and particularly page 229, col. 1 through page 232, col. 1). This process is referred to as intramolecular collision since it is believed that quenching is caused by the direct contact of the donor and acceptor moieties (See: Yaron et al.). As demonstrated in molecular beacon experiments, the donor and acceptor moieties attached to analog probe complexes need not have a substantial overlap between the emission of the donor moieties and the absorbance of the acceptor moieties. Without intending to be bound to this hypothesis, this data suggested that collision or contact operates as the primary mode of quenching in analog probe complexes. In another embodiment, it is a change in environment around the fluorescent probe which results in the change in fluorescence, this may or may not directly be the acceptor moiety.

Nonfluorescent Signaling:

As discussed above, not all signaling is achieved through a fluorescent signal on the probe complex. Many alternative examples are discussed herein and are known to one of skill in the art. Examples include MRI based techniques and binding based techniques, where the binding agent can either have a fluorescent moiety or catalyze a particular reaction or similar signaling event.

Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probing nucleobase sequence/target sequence combination is often found by the well known technique of fixing several of the aforementioned stringency factors and then determining the effect of varying a factor. The same stringency factors can be modulated to control the stringency of hybridization of analog probe complexes to target sequences, except that the hybridization of a PNA is fairly independent of ionic strength. Optimal stringency for an assay can be experimentally determined by examination of each stringency factor until the desired degree of discrimination is achieved.

Exemplary Applications for Using Some of the Various Embodiments:

Whether support bound or in solution, the methods, kits and compositions disclosed herein are useful for the rapid, sensitive, reliable and versatile detection of target sequences which are particular to organisms which might be found in food, beverages, water, pharmaceutical products, personal care products, dairy products or environmental samples. Preferred beverages include soda, bottled water, fruit juice, beer, wine or liquor products. The methods, kits and compositions disclosed herein will be particularly useful for the analysis of raw materials, equipment, products or processes used to manufacture or store food, beverages, water, pharmaceutical products, personal care products, dairy products or environmental samples.

Whether support bound or in solution, the methods, kits and compositions are also particularly useful for the rapid, sensitive, reliable and versatile detection of target sequences which are particular to organisms which might be found in clinical environments. Consequently, the methods, kits and compositions disclosed herein will be useful for the analysis of clinical specimens or equipment, fixtures or products used to treat humans or animals. For example, assays can be used to detect a target sequence that is specific for a genetically based disease or is specific for a predisposition to a genetically based disease. Non-limiting examples of diseases include, beta-Thalassemia, sickle cell anemia, Factor-V Leiden, cystic fibrosis and cancer related targets such as p53, p10, BRC-1 and BRC-2. The target sequence can be related to a chromosomal DNA, wherein the detection, identification or quantitation of the target sequence can be used in relation to forensic techniques such as prenatal screening, paternity testing, identity confirmation or crime investigation.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments.

### Incorporation By Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including but not limited to defined terms, term usage, described techniques, or the like, this application controls.

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A L-DNA/PNA analog probe complex comprising:
a first segment comprising a protein nucleic acid (PNA) segment, wherein said PNA segment comprises a PNA probe sequence that is capable of binding to a target sequence;
a fluorescent marker attached to the first segment; and
a second segment comprising a left-handed DNA (L-DNA) sequence that is capable of dissociably binding to said PNA probe sequence.

2. The L-DNA/PNA analog probe complex of claim 1, wherein the PNA segment further comprises a second PNA sequence that is configured to not hybridize to the L-DNA sequence, wherein said second PNA sequence binds to the target sequence.

3. The L-DNA/PNA analog probe complex of claim 2, wherein the first segment and the second segment have the same number of nucleotides.

4. The L-DNA/PNA analog probe complex of claim 1, wherein the first and the second segments are bound together via the hybridization of the first PNA sequence and the L-DNA sequence.

5. The L-DNA/PNA analog probe complex of claim 1, wherein the PNA probe sequence and the L-DNA sequence are complementary sequences.

6. The L-DNA/PNA analog probe complex of claim 1 further comprising a first fluorescent moiety as the fluorescent marker on the first segment and a second fluorescent moiety on the second segment, wherein hybridization of the first and second segments results in a first fluorescent signature from the first and second moieties, and a second fluorescent signature is obtained from the first and second moieties in the absence of hybridization.

7. The L-DNA/PNA analog probe complex of claim 6, wherein the first PNA sequence binds to a target sequence, wherein said target sequence is a RNA sequence.

8. The L-DNA/PNA analog probe complex of claim 7, wherein the first segment is longer in length than the second segment on an end of the first segment distal to the fluorescent moiety.

9. The L-DNA/PNA analog probe complex of claim 8, wherein the nucleotides in the L-DNA segment have a conformation of 1′S, 3′R, and 4′S.

10. A method of in-situ hybridization comprising:
contacting an analog probe complex with a sample comprising a target sequence, said analog probe complex comprising a) a first segment comprising a first PNA sequence, wherein said first PNA sequence will hybridize to a target sequence, b) a second segment, said second segment comprising a first L-DNA sequence, wherein said first L-DNA sequence can dissociably bind to said first PNA sequence, c) a fluorophore attached to the first segment, and d) a quencher attached to the second segment;
allowing the PNA sequence to bind to the target sequence;
fixing the sample; and
observing a fluorescence from the fluorophore.

11. The method of claim 10, wherein the PNA sequence is longer than the L-DNA sequence.

12. The method of claim 10, wherein the target sequence is mRNA.

13. The method of claim 10, wherein no wash step is performed after contacting an analog probe and after observing a fluorescence.

14. The method of claim 10, wherein a temperature of a mixture in which the contacting step occurs is lower than a temperature of a mixture that would be required for the PNA segment from an otherwise identical PNA/D-DNA analog probe complex to dissociate in the presence of a target.

15. The method of claim 10, wherein the fixing of the sample occurs before the analog probe complex is contacted with the sample.

16. A kit for wash free in situ hybridization comprising:
an analog probe complex comprising 1) a first section comprising a fluorescent marker and a PNA sequence, wherein said PNA sequence will hybridize to a target sequence and can dissociably bind to a L-DNA sequence, and 2) a second section comprising an L-DNA sequence and a quencher, wherein said L-DNA sequence can dissociably bind to said PNA sequence; and
a fixation reagent.

17. The kit of claim 16, further comprising a low ionic strength solution, wherein the low ionic strength is sufficient for dissociation of a L-DNA/PNA hybridized probe, but insufficient for effective dissociation of a D-DNA/PNA probe.

18. The kit of claim 16, wherein the fluorescent marker is a quantum dot.

19. An analog probe complex for the detection of nucleic acids, said analog probe complex comprising:
a first nucleic acid analog segment comprising a means for detecting a target sequence; and
a second nucleic acid analog segment comprising a means for detecting the second nucleic acid analog segment and a means for hybridizing said first segment with said second segment.
